(19) United States
(12) Patent Application Publication
(10) Pub. No.: US 2020/0253923 A1
(43) Pub. Date: Aug. 13, 2020

(54) NEW CANNABIS COMPOSITIONS AND INDUSTRIAL METHODS FOR PRODUCTION THEREOF

(71) Applicant: BUZZELET DEVELOPMENT AND TECHNOLOGIES LTD, Or-Akiva (IL)

(72) Inventor: Aharon EYAL, Jerusalem (IL)

(21) Appl. No.: 16/862,599

(22) Filed: Apr. 30, 2020

Related U.S. Application Data
(63) Continuation-in-part of application No. PCT/IB2018/058504, filed on Oct. 30, 2018.
(60) Provisional application No. 62/579,834, filed on Oct. 31, 2017.
(60) Provisional application No. 62/701,812, filed on Jul. 22, 2018.

(51) Int. Cl. A61K 31/352; A61K 31/05; A61K 9/20; A61K 9/00

(52) U.S. Cl. CPC A61K 31/352; A61K 9/0078; A61K 9/20; A61K 31/05

(57) ABSTRACT

Provided are homogeneous cannabis plant material compositions comprising tetrahydrocannabinol (THC) and cannabidiol (CBD) at specified concentrations, wherein the plant material is in the form of comminuted particles; wherein at least 70 wt % of the comminuted cannabis plant material is of a size greater than 0.3 and less than 4.2 millimeters; and wherein the composition comprises at least 1 wt % and less than 20 wt % moisture. Further provided are products comprising the compositions and methods of preparation thereof.

### FIELD OF THE INVENTION

The field of art to which this invention generally pertains is cannabis compositions, and more specifically to new cannabinoid compositions and industrial methods for production thereof.

### BACKGROUND OF THE INVENTION

Cannabis is a complex plant comprising over 400 chemical entities, some of which have opposing pharmacological effects. The main subspecies are Cannabis indica and Cannabis sativa, of which Cannabis sativa is the more commonly known.

The main active components of cannabis are named cannabinoids. So far, more than 100 cannabinoids have been discovered. Individual cannabis plants may contain multiple cannabinoids, sometimes dozens of them. The best known cannabinoids are tetrahydrocannabinol (THC) and cannabidiol (CBD). Others include cannabigerol (CBG), cannabichromene (CBC) tetrahydrocannabivarin (THCV) Cannabidivarin (CBDV) and cannabinol (CBN). Cannabinoids are found in various parts of the cannabis plant. Typically, their concentration is greatest in the flower (the bud), particularly in the trichomes.

In the naturally occurring cannabis plant, these cannabinoids are mostly synthesized and accumulated in their acid form as cannabinoid acids such as CBDa and THCa. For most therapeutic applications, cannabinoids are heat treated to effect thermal decarboxylation of the acid form of the cannabinoids into the decarboxylated form, such as CBD or THC.

Cannabis plants also include other components, e.g. terpenes and falvenoids. As used here, the term terpenes also include terpenoids. Terpenes are believed to affect the mechanism of operation and therapeutic effect of the cannabinoids. Known cannabis terpenes include pinene, limonene, linalool, caryophyllene, myrcene, humulene, borneol, eucalyptol, terpineol, nerolidol, phytol, geraniol, bisabolol and camphene.

There are hundreds of cannabis plant strains, which differ in many aspects, including their cannabinoids content. For example, some have a high concentration of THC (more than 20 wt %), while others have high CBD concentrations. There are also major differences in the terpene content.

Therapeutic cannabis is used in various forms, e.g. smoking, vaporizers, oils, ointments, pills, cookies, drinks, sprays, including nasal sprays, etc. In vaporizers, plant material, oil or other forms are heated to a suitable temperature and for a suitable time in order for decarboxylation and evaporation to occur and the patient inhales the formed vapors.

Therapeutic use requires high standard of manufacture, high product quality, accuracy in dosing and repeatability, so that the patient gets each time the compositions found most suitable for her or him.

Regulations in many countries are changing these days to allow therapeutic use of cannabis and the number of treated patients is already many millions. There is therefore a need for industrial production generating products at best quality standards.

The vast majority of patients smoke their medical cannabis, which in current practice limits the compositions available for administration to those present in known strains of cannabis plants as grown. This presents a challenge to the treatment. For example, there are many strains with various THC contents and minimal CBD content, as well as strains with high CBD content and minimal THC content, but there is only limited availability of strains with THC to CBD weight/weight ratio of 5/1, 4/1, 3/1, 2/1, 1/1, 1/2, 1/3, 1/4 or 1/5. Various studies have shown that such compositions having such currently unavailable ratios may potentially the best ones for treating various indications. Alternatively or additionally, such currently unavailable compositions are desired for specific stages of a treatment, e.g. the starting-up phase, or at particular hours of the day. For example, the Israeli Ministry of Health has selected ii medical cannabis compositions as recommended for use in therapy and has determined which one to be used at various stages of treatment. A major fraction of those compositions cannot be provided by currently known strains. Development and cultivation of new strains to suit those required compositions might be feasible, but costly and time consuming. New cultivation practices may have to be developed and adopted at additional cost. Additionally, there are compositions that even newly developed strains could probably not provide. Those include compositions where total cannabinoid concentration is high, e.g. higher than 30%. FIG. 1 (prior art) is a table showing compositions which are currently available in known strains of cannabis plants.

Hence, there is a strong need for cannabis plant products having compositions comprising CBD and THC concentrations and/or ratios that are currently unavailable, that are reliable, accurate and suitable for use by treated patients. There is also a need for methods to produce such products on an industrial scale and at an industrial production rate.

### SUMMARY OF THE INVENTION

According to an aspect of some embodiments of the present invention, there is provided a homogeneous cannabis plant material composition comprising tetrahydrocannabinol (THC) and cannabidiol (CBD), wherein the plant material is in the form of comminuted particles and wherein
(i) tetrahydrocannabinol and cannabidiol concentrations are selected from the group consisting of
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) more than 36 wt % tetrahydrocannabinol and 0.01-3 wt % cannabidiol;
(u) more than 36 wt % tetrahydrocannabinol and 3-6 wt % cannabidiol; and
(v) more than 36 wt % tetrahydrocannabinol and more than 6 wt % cannabidiol; and wherein
(ii) at least 70 wt % of the comminuted cannabis plant material is of a size greater than 0.3 millimeter and less than 4.2 millimeters;
(iii) the composition comprises at least 1 wt % moisture and less than 20 wt % moisture;
(iv) the homogeneous composition, when separated into at least a 10 gram sample, contains a tetrahydrocannabinol concentration in a 1 gram fraction of the at least 10 gram sample within 15% of the tetrahydrocannabinol concentration in a separate 1 gram fraction of said at least 10 gram sample; and
(v) and the homogeneous composition, when separated into at least a 10 gram sample, contains a cannabidiol concentration in a 1 gram fraction of the at least 10 gram sample within 15% of the cannabidiol concentration in a separate 1 gram fraction of said at least 10 gram sample.

According to a further aspect of some embodiments of the present invention, there is provided a homogeneous tablet, comprising tetrahydrocannabinol (THC) and cannabidiol (CBD), wherein
(i)tetrahydrocannabinol and cannabidiol and cannabidiol concentrations are:
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) More than 36 wt % tetrahydrocannabinol and 0.01-3 wt % cannabidiol;
(u) More than 36 wt % tetrahydrocannabinol and 3-6 wt % cannabidiol; or
(v) More than 36 wt % tetrahydrocannabinol and more than-6 wt % cannabidiol;
(ii) said tablet moisture content is greater than 1 wt % and less than 20 wt %;
(iii) said tablet bulk density is at least 0.1 gram/milliliter and less than 1.2 gram/milliliter;
(iv) the tetrahydrocannabinol concentration in a fraction of said tablet, is within 15% of the tetrahydrocannabinol concentration in a separate fraction of said tablet;
(v) the cannabidiol concentration in a fraction of said tablet, is within 15% of the cannabidiol concentration in a separate fraction of said tablet; and
(vi) said tablet comprises at least 70 wt % cannabis plant material.

According to a further aspect of some embodiments of the present invention, there is provided a method for producing the homogeneous cannabis plant material composition as disclosed herein, the method comprising
(i) providing a tetrahydrocannabinol-comprising cannabis plant material;
(ii) providing a cannabidiol-comprising cannabis plant material;
(iii) (a) comminuting said tetrahydrocannabinol-comprising cannabis plant materials, and said cannabidiol-comprising cannabis plant material whereby two separate comminuted homogeneous cannabis plant materials are formed, and/or
(b) comminuting one of said tetrahydrocannabinol-comprising cannabis plant materials, and said cannabidiol-comprising cannabis plant material, whereby a comminuted homogeneous cannabis plant material and a non-comminuted cannabis plant material are obtained and extracting the non-comminuted cannabis plant material to form a cannabinoid-comprising extract; and
(iv) (a) combining said two separate comminuted homogeneous cannabis plant materials of step (iii)(a) in a proportion calculated to provide said tetrahydrocannabinol and cannabidol concentrations of said composition, and/or (b) combining said comminuted homogeneous cannabis plant material and said cannabinoid-comprising extract of step (iii)(b) in a proportion calculated to provide the homogeneous comminuted cannabis plant material composition as disclosed herein.

Additional embodiments include: the method described above wherein said comminuting comprises
(i) maintaining said provided cannabis plant material at a temperature of less than 0° C. for at least 1 hour to form a frozen cannabis plant material;
(ii) comminuting said frozen cannabis plant material by pressing against a mesh screen with a sieve size smaller than 4.2 millimeters to form expelled homogeneous composition comprising comminuted cannabis plant material; and collecting said expelled, homogeneous composition;
the method described above wherein said extracting comprises optionally comminuting the non-comminuted cannabis plant material of step (iii)(b), whereby comminuted cannabis plant material is formed;
contacting said non-comminuted cannabis plant material or said comminuted cannabis plant material with a liquid extractant for at least 1 minute to form cannabinoid-depleted plant material and a cannabinoid-comprising extractant, and separating said cannabinoid-depleted plant material from said cannabinoid-comprising extractant to form separated cannabinoid-comprising extractant; the method described above comprising comminuting both said cannabis plant materials and blending said two comminuted cannabis plant materials with said cannabinoid-depleted plant material in a proportion calculated to form the said composition; the method described above comprising blending said comminuted cannabis plant material and said cannabinoid-comprising extract with said cannabinoid-depleted plant material in a proportion calculated to form the said composition; the method described above wherein at least 70 wt % of said provided tetrahydrocannabinol-comprises cannabis plant material and/or said provided cannabidiol-comprising cannabis plant material is derived from cannabis flower; the method described above wherein said screen pore size is at least 0.3 millimeter; the method described above wherein said provided tetrahydrocannabinol-comprising cannabis plant material, said provided cannabidiol-comprising cannabis plant material or both comprise less than 30% by weight moisture; the method described above wherein said provided tetrahydrocannabinol-comprising cannabis plant material, said provided cannabidiol-comprising cannabis plant material or both comprise moisture at more than 1% by weight; the method described above further comprising adding said homogeneous composition to a cigarettes tube or a cigarette paper, whereby a cigarette is formed; the method described above further comprising pressing said homogeneous comminuted composition to form a homogeneous cannabis tablet.

A cannabis cigarette produced according to the method described above is also described wherein said cigarette contains a top fraction and a bottom fraction and the concentration of tetrahydrocannabinol in a 0.1 gram sample from the top fraction of the cigarette is within 15% of tetrahydrocannabinol concentration in a 0.1 gram sample from the bottom fraction of the cigarette and wherein cannabidiol in a 0.1 gram sample from the top fraction of the cigarette is within 15% of cannabidiol concentration in a 0.1 gram sample from the bottom fraction of the cigarette.

A batch of cannabis cigarettes produced according to the method described above is also described wherein the content of tetrahydrocannabinol in one third of the batch of the cigarettes is within 15% of the tetrahydrocannabinol content in a separate third of the batch of the cigarettes and wherein the content of cannabidiol in one third of the batch of the cigarettes is within 15% of the cannabidiol content in a separate third of the batch of the cigarettes.

A homogeneous cannabis tablet produced according to the method described above is also described wherein said tablet has a bulk density of at least 0.1 gram/milliliter.

Additional embodiments include: the homogeneous cannabis tablet produced according to the method described above wherein the tablet has a bulk density of less than 1.2 gram/milliliter; and a homogeneous cannabis tablet produced according to the method described above wherein the concentration of tetrahydrocannabinol in a fraction of said homogeneous cannabis tablet is within 15% of the tetrahydrocannabinol concentration in a separate fraction of the tablet and wherein the concentration of cannabidiol in a fraction of said homogeneous cannabis tablet is within 15% of the cannabidiol concentration in a separate fraction of the tablet.

### BRIEF DESCRIPTION OF THE FIGURES

FIG. 1 (prior art) is a table showing compositions which are currently available in known strains of cannabis plants.

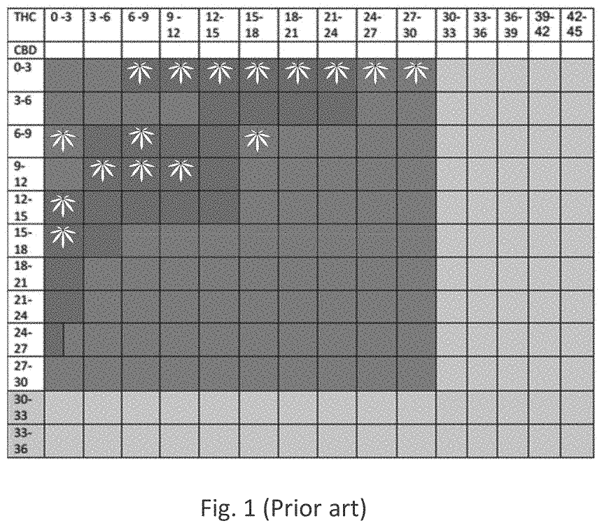

### DETAILED DESCRIPTION OF THE INVENTION

The present invention, in at least some embodiments, relates to a homogeneous cannabis plant material composition comprising tetrahydrocannabinol (THC) and cannabidiol (CBD), wherein the plant material is in the form of comminuted particles and wherein tetrahydrocannabinol and cannabidiol are present in specified concentrations which are not known in the art.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. As used in the description of the invention and the appended claims, the singular forms “a,” “an,” and “the” are intended to include the plural forms as well, unless the context clearly indicates otherwise.

Unless indicated otherwise, percent is weight percent (%wt).

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed.

Provided is homogeneous cannabis plant material composition comprising tetrahydrocannabinol (THC) and cannabidiol (CBD), wherein the plant material is in the form of comminuted particles and wherein
(i) tetrahydrocannabinol and cannabidiol concentrations are selected from the group consisting of
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) more than 36 wt % tetrahydrocannabinol and 0.01-3 wt % cannabidiol;
(u) more than 36 wt % tetrahydrocannabinol and 3-6 wt % cannabidiol; and
(v) more than 36 wt % tetrahydrocannabinol and more than6 wt % cannabidiol; and wherein
(ii) at least 70 wt % of the comminuted cannabis plant material is of a size greater than 0.3 millimeter and less than 4.2 millimeters;
(iii) the composition comprises at least 1 wt % moisture and less than 20 wt % moisture;
(iv) the homogeneous composition, when separated into at least a 10 gram sample, contains a tetrahydrocannabinol concentration in a 1 gram fraction of the at least 10 gram sample within 15% of the tetrahydrocannabinol concentration in a separate 1 gram fraction of said at least 10 gram sample; and
(v) and the homogeneous composition, when separated into at least a 10 gram sample, contains a cannabidiol concentration in a 1 gram fraction of the at least 10 gram sample within 15% of the cannabidiol concentration in a separate 1 gram fraction of said at least 10 gram sample.

According to an embodiment, said homogeneous comminuted cannabis plant material composition comprises 0.01-6 wt % tetrahydrocannabinol and 0.01-6 wt % cannabidiol.

According to an embodiment, said homogeneous composition comprises at least 0.01 wt % tetrahydrocannabinol, at least 2 wt % or at least 4 wt %. According to an embodiment, said homogeneous composition comprises less than 6 wt % tetrahydrocannabinol, less than 4 wt % or less than 2 wt %. According to an embodiment, said homogeneous composition comprises at least 0.1 wt % cannabidiol, at least 2 wt % or at least 4 wt %.

According to an embodiment, said homogeneous composition comprises less than 6 wt % cannabidiol, less than 4 wt % or less than 2 wt %.

According to an embodiment, said homogeneous comminuted cannabis plant material composition comprises 0.01-6 wt % tetrahydrocannabinol and 6-12 wt % cannabidiol.

According to an embodiment, said homogeneous composition comprises at least 0.01 wt % tetrahydrocannabinol, at least 2 wt % or at least 4 wt %.

According to an embodiment, said homogeneous composition comprises less than 6 wt % tetrahydrocannabinol, less than 4 wt % or less than 2 wt %. According to an embodiment, said homogeneous composition comprises at least 6 wt % cannabidiol, at least 8 wt % or at least 10 wt %.

According to an embodiment, said homogeneous composition comprises less than 12 wt % cannabidiol, less than 10 wt % or less than 8 wt %.

According to an embodiment, said homogeneous comminuted cannabis plant material composition comprises 0.01-6 wt % tetrahydrocannabinol and 12-18 wt % cannabidiol.

According to an embodiment, said homogeneous composition comprises at least 0.01 wt % tetrahydrocannabinol, at least 2 wt % or at least 4 wt %.

According to an embodiment, said homogeneous composition comprises less than 6 wt % tetrahydrocannabinol, less than 4 wt % or less than 2 wt %.

According to an embodiment, said homogeneous composition comprises at least 12 wt % cannabidiol, at least 14 wt % or at least 16 wt %.

According to an embodiment, said homogeneous composition comprises less than 18 wt % cannabidiol, less than 16 wt % or less than 14 wt %.

According to an embodiment, said homogeneous comminuted cannabis plant material composition comprises 0.01-6 wt % tetrahydrocannabinol and 18-24 wt % cannabidiol.

According to an embodiment, said homogeneous composition comprises at least 0.01 wt % tetrahydrocannabinol, at least 2 wt % or at least 4 wt %.

According to an embodiment, said homogeneous composition comprises less than 6 wt % tetrahydrocannabinol, less than 4 wt % or less than 2 wt %.

According to an embodiment, said homogeneous composition comprises at least 18 wt % cannabidiol, at least 20 wt % or at least 22 wt %.

According to an embodiment, said homogeneous composition comprises less than 24 wt % cannabidiol, less than 22 wt % or less than 20 wt %.

According to an embodiment, said homogeneous comminuted cannabis plant material composition comprises 0.01-6 wt % tetrahydrocannabinol and more than 24 wt % cannabidiol.

According to an embodiment, said homogeneous composition comprises at least 0.01 wt % tetrahydrocannabinol, at least 2 wt % or at least 4 wt %.

According to an embodiment, said homogeneous composition comprises less than 6 wt % tetrahydrocannabinol, less than 4 wt % or less than 2 wt %.

According to an embodiment, said homogeneous composition comprises at least 24 wt % cannabidiol, at least 26 wt %, at least28 wt % or at least 30 wt %.

According to an embodiment, said homogeneous comminuted cannabis plant material composition comprises 6-12 wt % tetrahydrocannabinol and 0.01-6 wt % cannabidiol.

According to an embodiment, said homogeneous composition comprises at least 6 wt % tetrahydrocannabinol, at least 8 wt % or at least 10 wt %.

According to an embodiment, said homogeneous composition comprises less than 12 wt % tetrahydrocannabinol, less than 10 wt % or less than 8 wt %.

According to an embodiment, said homogeneous composition comprises at least 0.01 wt % cannabidiol, at least 2 wt % or at least 4 wt %.

According to an embodiment, said homogeneous composition comprises less than 6 wt % cannabidiol, less than 4 wt % or less than 2 wt %.

According to an embodiment, said homogeneous comminuted cannabis plant material composition comprises 6-12 wt % tetrahydrocannabinol and 6-12 wt % cannabidiol.

According to an embodiment, said homogeneous composition comprises at least 6 wt % tetrahydrocannabinol, at least 8 wt % or at least 10 wt %.

According to an embodiment, said homogeneous composition comprises less than 12 wt % tetrahydrocannabinol, less than 10 wt % or less than 8 wt %.

According to an embodiment, said homogeneous composition comprises at least 6 wt % cannabidiol, at least 8 wt % or at least 10 wt %.

According to an embodiment, said homogeneous composition comprises less than 12 wt % cannabidiol, less than 10 wt % or less than 8 wt %.

According to an embodiment, said homogeneous comminuted cannabis plant material composition comprises 6-12 wt % tetrahydrocannabinol and 12-18 wt % cannabidiol.

According to an embodiment, said homogeneous composition comprises at least 6 wt % tetrahydrocannabinol, at least 8 wt % or at least 10 wt %.

According to an embodiment, said homogeneous composition comprises less than 12 wt % tetrahydrocannabinol, less than 10 wt % or less than 8 wt %.

According to an embodiment, said homogeneous composition comprises at least 12 wt % cannabidiol, at least 14 wt % or at least 16 wt %.

According to an embodiment, said homogeneous composition comprises less than 18 wt % cannabidiol, less than 16 wt % or less than 14 wt %.

According to an embodiment, said homogeneous comminuted cannabis plant material composition comprises 6-12 wt % tetrahydrocannabinol and 18-24 wt % cannabidiol.

According to an embodiment, said homogeneous composition comprises at least 6 wt % tetrahydrocannabinol, at least 8 wt % or at least 10 wt %.

According to an embodiment, said homogeneous composition comprises less than 12 wt % tetrahydrocannabinol, less than 10 wt % or less than 8 wt %. According to an embodiment, said homogeneous composition comprises at least 18 wt % cannabidiol, at least 20 wt % or at least 22 wt %.

According to an embodiment, said homogeneous composition comprises less than 24 wt % cannabidiol, less than 22 wt % or less than 20 wt %.

According to an embodiment, said homogeneous comminuted cannabis plant material composition comprises 6-12 wt % tetrahydrocannabinol and more than 24 wt % cannabidiol.

According to an embodiment, said homogeneous composition comprises at least 6 wt % tetrahydrocannabinol, at least 8 wt % or at least 10 wt %. According to an embodiment, said homogeneous composition comprises less than 12 wt % tetrahydrocannabinol, less than 10 wt % or less than 8 wt %.

According to an embodiment, said homogeneous composition comprises at least 24 wt % cannabidiol, at least 26 wt %, at least28 wt % or at least 30 wt %.

According to an embodiment, said homogeneous comminuted cannabis plant material composition comprises 12-18 wt % tetrahydrocannabinol and 0.01-6 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 0.1 wt % cannabidiol, at least 2 wt % or at least 4 wt %. According to an embodiment, said homogeneous composition comprises less than 6 wt % cannabidiol, less than 4 wt % or less than 2 wt %.

According to an embodiment, said homogeneous comminuted cannabis plant material composition comprises 12-18 wt % tetrahydrocannabinol and 6-12 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 6 wt % cannabidiol, at least 8 wt % or at least 10 wt %. According to an embodiment, said homogeneous composition comprises less than 12 wt % cannabidiol, less than 10 wt % or less than 8 wt %.

According to an embodiment, said homogeneous comminuted cannabis plant material composition comprises 12-18 wt % tetrahydrocannabinol and 12-18 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 12 wt % cannabidiol, at least 14 wt % or at least 16 wt %. According to an embodiment, said homogeneous composition comprises less than 18 wt % cannabidiol, less than 16 wt % or less than 14 wt %.

According to an embodiment, said homogeneous comminuted cannabis plant material composition comprises 12-18 wt % tetrahydrocannabinol and 18-24 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 18 wt % cannabidiol, at least 20 wt % or at least 22 wt %. According to an embodiment, said homogeneous composition comprises less than 24 wt % cannabidiol, less than 22 wt % or less than 20 wt %.

According to an embodiment, said homogeneous comminuted cannabis plant material composition comprises 12-18 wt % tetrahydrocannabinol and more than 24 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 24 wt % cannabidiol, at least 26 wt %, at least28 wt % or at least 30 wt %.

According to an embodiment, said homogeneous comminuted cannabis plant material composition comprises 18-24 wt % tetrahydrocannabinol and 0.01-6 wt % cannabidiol. According to an embodiment, said homogeneous composition comprises at least 18 wt % tetrahydrocannabinol, at least 20 wt % or at least 22 wt %. According to an embodiment, said homogeneous composition comprises less than 24 wt % tetrahydrocannabinol, less than 22 wt % or less than 20 wt %. According to an embodiment, said homogeneous composition comprises at least 0.1 wt % cannabidiol, at least 2 wt % or at least 4 wt %. According to an embodiment, said homogeneous composition comprises less than 6 wt % cannabidiol, less than 4 wt % or less than 2 wt %.

According to an embodiment, said homogeneous comminuted cannabis plant material composition comprises 18-24 wt % tetrahydrocannabinol and 6-12 wt % cannabidiol. According to an embodiment, said homogeneous composition comprises at least 18 wt % tetrahydrocannabinol, at least 20 wt % or at least 22 wt %. According to an embodiment, said homogeneous composition comprises less than 24 wt % tetrahydrocannabinol, less than 22 wt % or less than 20 wt %. According to an embodiment, said homogeneous composition comprises at least 6 wt % cannabidiol, at least 8 wt % or at least 10 wt %. According to an embodiment, said homogeneous composition comprises less than 12 wt % cannabidiol, less than 10 wt % or less than 8 wt %.

According to an embodiment, said homogeneous comminuted cannabis plant material composition comprises 18-24 wt % tetrahydrocannabinol and 12-18 wt % cannabidiol. According to an embodiment, said homogeneous composition comprises at least 18 wt % tetrahydrocannabinol, at least 20 wt % or at least 22 wt %. According to an embodiment, said homogeneous composition comprises less than 24 wt % tetrahydrocannabinol, less than 22 wt % or less than 20 wt %. According to an embodiment, said homogeneous composition comprises at least 12 wt % cannabidiol, at least 14 wt % or at least 16 wt %. According to an embodiment, said homogeneous composition comprises less than 18 wt % cannabidiol, less than 16 wt % or less than 14 wt %.

According to an embodiment, said homogeneous comminuted cannabis plant material composition comprises 18-24 wt % tetrahydrocannabinol and 18-24 wt % cannabidiol. According to an embodiment, said homogeneous composition comprises at least 18 wt % tetrahydrocannabinol, at least 20 wt % or at least 22 wt %. According to an embodiment, said homogeneous composition comprises less than 24 wt % tetrahydrocannabinol, less than 22 wt % or less than 20 wt %. According to an embodiment, said homogeneous composition comprises at least 18 wt % cannabidiol, at least 20 wt % or at least 22 wt %. According to an embodiment, said homogeneous composition comprises less than 24 wt % cannabidiol, less than 22 wt % or less than 20 wt %. According to an embodiment, said homogeneous comminuted cannabis plant material composition comprises 18-24 wt % tetrahydrocannabinol and more than 24 wt % cannabidiol.

According to an embodiment, said homogeneous composition comprises at least 18 wt % tetrahydrocannabinol, at least 20 wt % or at least 22 wt %. According to an embodiment, said homogeneous composition comprises less than 24 wt % tetrahydrocannabinol, less than 22 wt % or less than 20 wt %. According to an embodiment, said homogeneous composition comprises at least 24 wt % cannabidiol, at least 26 wt %, at least28 wt % or at least 30 wt %.

According to an embodiment, said homogeneous comminuted cannabis plant material composition comprises more than 24 wt % tetrahydrocannabinol and 0.01-6 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 0.1 wt % cannabidiol, at least 2 wt % or at least 4 wt %. According to an embodiment, said homogeneous composition comprises less than 6 wt % cannabidiol, less than 4 wt % or less than 2 wt %.

According to an embodiment, said homogeneous comminuted cannabis plant material composition comprises more than 24 wt % tetrahydrocannabinol and 6-12 wt % cannabidiol. According to an embodiment, said homogeneous composition comprises at least 24 wt % tetrahydrocannabinol, at least 26 wt %, at least 28 wt % or at least 30 wt %. According to an embodiment, said homogeneous composition comprises at least 6 wt % cannabidiol, at least 8 wt % or at least 10 wt %. According to an embodiment, said homogeneous composition comprises less than 12 wt % cannabidiol, less than 10 wt % or less than 8 wt %.

According to an embodiment, said homogeneous comminuted cannabis plant material composition comprises more than 24 wt % tetrahydrocannabinol and 12-18 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 12 wt % cannabidiol, at least 14 wt % or at least 16 wt %. According to an embodiment, said homogeneous composition comprises less than 18 wt % cannabidiol, less than 16 wt % or less than 14 wt %.

According to an embodiment, said homogeneous comminuted cannabis plant material composition comprises more than 24 wt % tetrahydrocannabinol and 18-24 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 18 wt % cannabidiol, at least 20 wt % or at least 22 wt %. According to an embodiment, said homogeneous composition comprises less than 24 wt % cannabidiol, less than 22 wt % or less than 20 wt %.

According to an embodiment, said homogeneous comminuted cannabis plant material composition comprises more than 24 wt % tetrahydrocannabinol and more than 24 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 24 wt % cannabidiol, at least 26 wt %, at least28 wt % or at least 30 wt %.

According to an embodiment, said homogeneous comminuted cannabis plant material composition comprises 24-30 wt % tetrahydrocannabinol and 0.01-6 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 0.01 wt % cannabidiol, at least 2 wt %, at least4 wt % or at least 6 wt %.

According to an embodiment, said homogeneous comminuted cannabis plant material composition comprises 24-30 wt % tetrahydrocannabinol and 6-12 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 6 wt % cannabidiol, at least 8 wt %, at least 10 wt % or at least 12 wt %.

According to an embodiment, said homogeneous comminuted cannabis plant material composition comprises 24-30 wt % tetrahydrocannabinol and 12-18 wt % cannabidiol. According to an embodiment, said homogeneous composition comprises at least 24 wt % tetrahydrocannabinol, at least 26 wt %, at least28 wt % or at least 30 wt %. According to an embodiment, said homogeneous composition comprises at least 12 wt % cannabidiol, at least 14 wt %, at least 16 wt % or at least 18 wt %.

According to an embodiment, said homogeneous comminuted cannabis plant material composition comprises 30-36 wt % tetrahydrocannabinol and 0.01-6 wt % cannabidiol. According to an embodiment, said homogeneous composition comprises at least 30 wt % tetrahydrocannabinol, at least 32 wt %, at least 34 wt % or at least 36 wt %. According to an embodiment, said homogeneous composition comprises at least 0.01 wt % cannabidiol, at least 2 wt %, at least4 wt % or at least 6 wt %.

According to an embodiment, said homogeneous comminuted cannabis plant material composition comprises 30-36 wt % tetrahydrocannabinol and 6-12 wt % cannabidiol. According to an embodiment, said homogeneous composition comprises at least 30 wt % tetrahydrocannabinol, at least 32 wt %, at least 34 wt % or at least 36 wt %. According to an embodiment, said homogeneous composition comprises at least 6 wt % cannabidiol, at least 8 wt %, at least 10 wt % or at least 12 wt %.

According to an embodiment, said homogeneous comminuted cannabis plant material composition comprises 30-36 wt % tetrahydrocannabinol and 12-18 wt % cannabidiol. According to an embodiment, said homogeneous composition comprises at least 30 wt % tetrahydrocannabinol, at least 32 wt %, at least 34 wt % or at least 36 wt %. According to an embodiment, said homogeneous composition comprises at least 12 wt % cannabidiol, at least 14 wt %, at least 16 wt % or at least 18 wt %.

According to an embodiment, said homogeneous comminuted cannabis plant material composition comprises more than 36 wt % tetrahydrocannabinol and 0.01-6 wt % cannabidiol. According to an embodiment, said homogeneous composition comprises at least 0.01 wt % cannabidiol, at least 2 wt %, at least4 wt % or at least 6 wt %.

According to an embodiment, said homogeneous comminuted cannabis plant material composition comprises more than 36 wt % tetrahydrocannabinol and 6-12 wt % cannabidiol. According to an embodiment, said homogeneous composition comprises at least 6 wt % cannabidiol, at least 8 wt %, at least 10 wt % or at least 12 wt %.

According to an embodiment, said homogeneous comminuted cannabis plant material composition comprises more than 36 wt % tetrahydrocannabinol and 12-18 wt % cannabidiol. According to an embodiment, said homogeneous composition comprises at least 12 wt % cannabidiol, at least 14 wt %, at least 16 wt % or at least 18 wt %.

According to an embodiment, said homogeneous comminuted cannabis plant material composition comprises more than 36 wt % tetrahydrocannabinol and more then 6 wt % cannabidiol.

As used herein, the term cannabis plant material refers to material resulting from cannabis plant, e.g. portions of cannabis plant as such, and products of processing such portions of cannabis plant, by drying and curing wherein only water and some terpenes are removed while all other components of the cannabis plant are retained. According to an embodiment at least 70 wt % of said composition comprises cannabis plant material, at least 75 wt %, least 80 wt %, least 85 wt %, least 90 wt % or at least 95 wt %.

According to an embodiment at least 70 wt % of the cannabis plant material in said composition is derived from cannabis flower (also referred to as cannabis bud), at least 75 wt %, least 80 wt %, least 85 wt %, least 90 wt % or at least 95 wt %. As used herein, derived from cannabis flower refers to being cannabis flower or being a product or processing cannabis flower, e.g. drying and/or comminuting a cannabis flower. According to an embodiment, at least a fraction of said cannabis plant material in said composition comprises dried cannabis plant material, cured cannabis plant material or dried and cured cannabis plant material.

According to an embodiment said composition comprises comminuted cannabis plant material. As used herein the term comminuted material refers to cannabis plant material which has been processed such as by crushing or grinding to obtain a collection of small particles. According to an embodiment at least 70 wt % of the particles, at least 80 wt %, at least 90 wt % or at least 95 wt %, are of a size greater than 0.3 millimeter (mm), greater than 0.5 mm, greater than 0.7 mm, greater than 0.9 mm, greater than 1.1 mm or greater than 1.3 mm. As used herein, particle size refers to the largest dimension of said particles. According to an embodiment at least 70 wt % of the particles, at least 80 wt %, at least 90 wt % or at least 95 wt % are of a size less than 4.2 mm, less than 4.0 mm, less than 3.8 mm, less than 3.6 mm less than 3.4 mm, less than 3.2 mm, less than 3.0 mm, less than 2.6 mm or less than 2.4.

According to an embodiment, said homogeneous comminuted cannabis plant material composition comprises the cannabinoids tetrahydrocannabinol (THC) and cannabidiol (CBD). According to an embodiment, said homogeneous composition comprises at least three cannabinoids, at least four, at least five or at least six. As known in the art, cannabinoids have an acid form and a non-acid form (which is also referred to as decarboxylated form, since it can be generated by decarboxylating the acid form). The acid form is indicated herein by the letter (a) at the end of the cannabinoid acronym, e.g. tetrahydrocannabinolic acid is indicated as THCa, while the decarboxylated form is THC. As used herein, unless specified otherwise, the term cannabinoid and the name of the cannabinoid refers to both the acid form and the decarboxylated form. For example, tetrahydrocannabinol refers to THC and/or THCa and cannabidiol refers to CBDa and/or CBD. According to an embodiment, said homogeneous composition further comprises cannabigerol, cannabichromene tetrahydrocannabivarin, cannabidivarin and cannabinol and combinations thereof. According to an embodiment, said homogeneous composition of claim 1, comprises less than 1 wt % cannabinol, less than 0.8 wt %, less than 0.7 wt %, less than 0.6 wt %, less than 0.5 wt % or less than 0.1 wt %.

According to an embodiment, said homogeneous composition comprises more than 36 wt % tetrahydrocannabinol and 0.01-9 wt % cannabidiol. According to an embodiment, said homogeneous composition comprises at least 36 wt % tetrahydrocannabinol. According to an embodiment, said homogeneous composition comprises at least 0.01 wt % cannabidiol, at least 3 wt %, at least 5% or at least 9 wt %. According to an embodiment, said homogeneous composition comprises less than 9 wt % cannabidiol, less than 5 wt % or less than 3 wt %.

According to an embodiment, said homogeneous composition comprises 30-36 wt % tetrahydrocannabinol and 0.01-15 wt % cannabidiol. According to an embodiment, said homogeneous composition comprises at least 32 wt % tetrahydrocannabinol, at least 34 wt % or at least 36 wt %. According to an embodiment, said homogeneous composition comprises less than 36 wt % tetrahydrocannabinol, less than 34 wt % or less than 32 wt %. According to an embodiment, said homogeneous composition comprises at least 0.01 wt % cannabidiol, at least 7 wt % or at least 15 wt %. According to an embodiment, said homogeneous composition comprises less than 15 wt % cannabidiol, less than 10 wt % or less than 3 wt %.

According to an embodiment, said homogeneous composition comprises 24-30 wt % tetrahydrocannabinol and 0.01-18 wt % cannabidiol. According to an embodiment, said homogeneous composition comprises at least 26 wt % tetrahydrocannabinol, at least 28 wt % or at least 30 wt %. According to an embodiment, said homogeneous composition comprises less than 30 wt % tetrahydrocannabinol, less than 28 wt % or less than 26 wt %. According to an embodiment, said homogeneous composition comprises at least 0.01 wt % cannabidiol, at least 10 wt % or at least 16 wt %. According to an embodiment, said homogeneous composition comprises less than 18 wt % cannabidiol, less than 10 wt % or less than 3 wt %.

According to an embodiment, said homogeneous composition comprises 18-24 wt % tetrahydrocannabinol and 0.01-18 wt % cannabidiol. According to an embodiment, said homogeneous composition comprises at least 20 wt % tetrahydrocannabinol, at least 22 wt % or at least 24 wt %. According to an embodiment, said homogeneous composition comprises less than 26 wt % tetrahydrocannabinol, less than 24 wt % or less than 22 wt %. According to an embodiment, said homogeneous composition comprises at least 0.01 wt % cannabidiol, at least 10 wt % or at least 16 wt %. According to an embodiment, said homogeneous composition comprises less than 18 wt % cannabidiol, less than 10 wt % or less than 3 wt %.

According to an embodiment, said homogeneous composition comprises 12-18 wt % tetrahydrocannabinol and 0.01-21 wt % cannabidiol. According to an embodiment, said homogeneous composition comprises at least 12 wt % tetrahydrocannabinol, at least 14 wt % or at least 16 wt %. According to an embodiment, said homogeneous composition comprises less than 18 wt % tetrahydrocannabinol, less than 16 wt % or less than 14 wt %. According to an embodiment, said homogeneous composition comprises at least 0.01 wt % cannabidiol, at least 10 wt % or at least 20 wt %. According to an embodiment, said homogeneous composition comprises less than 21 wt % cannabidiol, less than 10 wt % or less than 3 wt %.

According to an embodiment, said homogeneous composition comprises 6-12 wt % tetrahydrocannabinol and 0.01-24 wt % cannabidiol. According to an embodiment, said homogeneous composition comprises at least 6 wt % tetrahydrocannabinol, at least 8 wt % or at least 10 wt %. According to an embodiment, said homogeneous composition comprises less than 12 wt % tetrahydrocannabinol, less than 10 wt % or less than 8 wt %. According to an embodiment, said homogeneous composition comprises at least 0.01 wt % cannabidiol, at least 10 wt % or at least 22 wt %. According to an embodiment, said homogeneous composition comprises less than 24 wt % cannabidiol, less than 10 wt % or less than 3 wt %.

According to an embodiment, said homogeneous composition comprises 0.01-6 wt % tetrahydrocannabinol and 0.01-34 wt % cannabidiol. According to an embodiment, said homogeneous composition comprises at least 0.01 wt % tetrahydrocannabinol, at least 2 wt % or at least 4 wt %. According to an embodiment, said homogeneous composition comprises less than 6 wt % tetrahydrocannabinol, less than 4 wt % or less than 2 wt %. According to an embodiment, said homogeneous composition comprises at least 0.01 wt % cannabidiol, at least 15 wt % or at least 30 wt %. According to an embodiment, said homogeneous composition comprises less than 34 wt % cannabidiol, less than 20 wt % or less than 10 wt %.

According to an embodiment, said homogeneous composition comprises 16-24 wt % tetrahydrocannabinol and 1-7 wt % cannabidiol. According to an embodiment, said homogeneous composition comprises at least 16 wt % tetrahydrocannabinol, at least 18 wt % or at least 20 wt %. According to an embodiment, said homogeneous composition comprises less than 24 wt % tetrahydrocannabinol, less than 22 wt % or less than 20 wt %. According to an embodiment, said homogeneous composition comprises at least 1 wt % cannabidiol, at least 3 wt % or at least 5 wt %. According to an embodiment, said homogeneous composition comprises less than 7 wt % cannabidiol, less than 5 wt % or less than 3 wt %.

According to an embodiment, said homogeneous composition comprises 11-18 wt % tetrahydrocannabinol and 0.5-5.5 wt % cannabidiol. According to an embodiment, said homogeneous composition comprises at least 11 wt % tetrahydrocannabinol, at least 13 wt % or at least 15 wt %. According to an embodiment, said homogeneous composition comprises less than 19 wt % tetrahydrocannabinol, less than 17 wt % or less than 15 wt %. According to an embodiment, said homogeneous composition comprises at least 0.5 wt % cannabidiol, at least 1.5 wt % or at least 2.5 wt %. According to an embodiment, said homogeneous composition comprises less than 5.5 wt % cannabidiol, less than 4.5 wt % or less than 3.5 wt %.

According to an embodiment, said homogeneous composition comprises 6-14 wt % tetrahydrocannabinol and 0.2-3.8 wt % cannabidiol. According to an embodiment, said homogeneous composition comprises at least 6 wt % tetrahydrocannabinol, at least 8 wt % or at least 10 wt %. According to an embodiment, said homogeneous composition comprises less than 14 wt % tetrahydrocannabinol, less than 12 wt % or less than 10 wt %. According to an embodiment, said homogeneous composition comprises at least 0.5 wt % cannabidiol, at least 1 wt % or at least 1.5 wt %. According to an embodiment, said homogeneous composition comprises less than 3.5 wt % cannabidiol, less than 2.5 wt % or less than 1.5 wt %.

According to an embodiment, said homogeneous composition comprises 6-14 wt % tetrahydrocannabinol and 6-14 wt % cannabidiol. According to an embodiment, said homogeneous composition comprises at least 6 wt % tetrahydrocannabinol, at least 8 wt % or at least 10 wt %. According to an embodiment, said homogeneous composition comprises less than 14 wt % tetrahydrocannabinol, less than 12 wt % or less than 10 wt %. According to an embodiment, said homogeneous composition comprises at least 6 wt % cannabidiol, at least 8 wt % or at least 10 wt %. According to an embodiment, said homogeneous composition comprises less than 14 wt % cannabidiol, less than 12 wt % or less than 10 wt %.

According to an embodiment, said homogeneous composition comprises 2.5-7.5 wt % tetrahydrocannabinol and 6-14 wt % cannabidiol. According to an embodiment, said homogeneous composition comprises at least 2.5 wt % tetrahydrocannabinol, at least 3.5 wt % or at least 4.5 wt %. According to an embodiment, said homogeneous composition comprises less than 7.5 wt % tetrahydrocannabinol, less than 6.5 wt % or less than 5.5 wt %. According to an embodiment, said homogeneous composition comprises at least 6 wt % cannabidiol, at least 8 wt % or at least 10 wt %. According to an embodiment, said homogeneous composition comprises less than 14 wt % cannabidiol, less than 12 wt % or less than 10 wt %.

According to an embodiment, said homogeneous composition comprises 0.5-5.5 wt % tetrahydrocannabinol and 11-19 wt % cannabidiol. According to an embodiment, said homogeneous composition comprises at least 0.5 wt % tetrahydrocannabinol, at least 1.5 wt % or at least 2.5 wt %. According to an embodiment, said homogeneous composition comprises less than 5.5wt % tetrahydrocannabinol, less than 4.5 wt % or less than 3.5 wt %. According to an embodiment, said homogeneous composition comprises at least 11 wt % cannabidiol, at least 13 wt % or at least 15 wt %. According to an embodiment, said homogeneous composition comprises less than 19 wt % cannabidiol, less than 17 wt % or less than 15 wt %.

According to an embodiment, said homogeneous composition comprises 0.1-2.5 wt % tetrahydrocannabinol and 16-24 wt % cannabidiol. According to an embodiment, said homogeneous composition comprises at least 0.5 wt % tetrahydrocannabinol, at least 1 wt % or at least 1.5 wt %. According to an embodiment, said homogeneous composition comprises less than 2.5 wt % tetrahydrocannabinol, less than 2 wt % or less than 1.5 wt %. According to an embodiment, said homogeneous composition comprises at least 16 wt % cannabidiol, at least 18 wt % or at least 20 wt %. According to an embodiment, said homogeneous composition comprises less than 24 wt % cannabidiol, less than 22 wt % or less than 20 wt %.

According to an embodiment, said homogeneous composition comprises 0.1-0.5 wt % tetrahydrocannabinol and 20-28 wt % cannabidiol. According to an embodiment, said homogeneous composition comprises at least 20 wt % cannabidiol, at least 22 wt % or at least 24 wt %. According to an embodiment, said homogeneous composition comprises less than 28 wt % cannabidiol, less than 26 wt % or less than 24 wt %.

According to an embodiment, the tetrahydrocannabinol comprises tetrahydrocannabinol and tetrahydrocannabinolic acid and the weight per weight ratio between tetrahydrocannabinol and tetrahydrocannabinolic acid in said composition is in the range between 20 to 1 and 1 to 20. According to an embodiment, the weight per weight ratio between tetrahydrocannabinol and tetrahydrocannabinolic acid in said composition is less than 18:1, less than 16:1, less than 14:1, less than 12:1, less than 10:1, less than 8:1, less than 6:1, less than 4:1, less than 2:1 or less than 1:1. According to an embodiment, the weight per weight ratio between tetrahydrocannabinol and tetrahydrocannabinolic acid in said composition is greater than 1:20, greater than 1:18, greater than 1:16, greater than 1:14, greater than 1:12, greater than 1:10, greater than 1:8, greater than 1:6, greater than 1:4, greater than 1:2, or greater than 1:1.

According to an embodiment, wherein the cannabinol comprises cannabinol and cannabinolic acid and the weight per weight ratio between cannabidiol and cannabidiolic acid in said composition is in the range between 20 to 1 and 1 to 20. According to an embodiment, the weight per weight ratio between cannabidiol and cannabidiolic acid in said composition is less than 18:1, less than 16:1, less than 14:1, less than 12:1, less than 10:1, less than 8:1, less than 6:1, less than 4:1, less than 2:1 or less than 1:1. According to an embodiment, the weight per weight ratio between cannabidiol and cannabidiolic acid in said composition is greater than 1:20, greater than 1:18, greater than 1:16, greater than 1:14, greater than 1:12, greater than 1:10, greater than 1:8, greater than 1:6, greater than 1:4, greater than 1:2, or greater than 1:1.

According to an embodiment said homogeneous composition further comprises at least one terpene, at least two, at least three, at least four or at least five terpenes. The term “terpene”, as used herein, refers to terpenes and/or terpenoids. According to an embodiment, said terpene is selected from the group consisting of pinene, limonene, linalool, caryophyllene, myrcene, humulene, borneol, eucalyptol, terpineol, nerolidol, phytol, geraniol, bisabolol, camphene, beta-amyrin, thujone, citronellol, pulegone, cycloartenol, isomers thereof and combinations thereof.

According to an embodiment said composition comprises moisture. As used herein, the term moisture refers to water contained in the plant material or wetting it. According to an embodiment, moisture content can be determined by loss on drying. According to an embodiment said composition comprises at least 1 wt % moisture; at least 3 wt %, at least 5 wt %, at least 7 wt %, at least 9 wt %, or at least 11 wt %. According to an embodiment said composition comprises less than 20 wt % moisture, less than 18 wt %, less than 16 wt %, or less than 14 wt %. According to an embodiment said composition comprises between 10 wt % and 14 wt % moisture or between 11 wt % and 13 wt % moisture.

Any amount of said composition is suitable. According to an embodiment, the amount of said composition is at least 100 gram (gr), at least 1 kilogram (Kg), at least 10 Kg, at least 100 Kg, or at least 1000 Kg.

According to an embodiment, said composition, is homogeneous. As used herein, the term homogeneous refers to having a similar content in various samples of the composition. According to an embodiment, a similar content means at least one of a similar size distribution and a similar concentration of at least one major component, at least two, at least three, at least four or at least five major components. As used herein, the term major component refers to a component comprising at least 1 wt % of said composition.

According to an embodiment, said major component is a the homogeneous composition, when separated into at least a 10 gram sample, contains a tetrahydrocannabinol concentration in a 1 gram fraction of the at least 10 gram sample, is within 15% of the cannabinoid concentration in a separate 1 gram fraction of said 10 gram sample within 13%, within 11%, within 9%, within 7%, within 5%, within 3%, within 2% or within 1%. According to an embodiment said cannabinoid comprises tetrahydrocannabinol (THC). According to another embodiment said cannabinoid comprises cannabidiol (CBD).

According to an embodiment, the size of at least 70 wt %, at least 80 wt % or at least 90 wt % of the comminuted cannabis plant material in a 1 gram fraction of at least 10 gram sample of said homogeneous composition, is within 15% of the size of at least 70 wt %, at least 80 wt % or at least 90 wt % of the comminuted cannabis plant material in a separate 1 gram fraction of said 10 gram sample, within 13%, within 11%, within 9%, within 7%, within 5%, within 3%, within 2% or within 1%.

According to an embodiment the homogeneous composition, when separated into at least a 10 gram sample, contains a tetrahydrocannabinol concentration in a 1 gram fraction of the at least 10 gram sample, is within 15% of the tetrahydrocannabinol concentration in a separate 1 gram fraction of said 10 gram sample, within 13%, within 11%, within 9%, within 7%, within 5%, within 3%, within 2% or within 1%. According to an embodiment, the cannabidiol concentration in a 1 gram fraction of at least 10 gram sample of said homogeneous composition, is within 15% of the cannabidiol concentration in a separate 1 gram fraction of said 10 gram sample, within 13%, within 11%, within 9%, within 7%, within 5%, within 3%, within 2% or within 1%. According to an embodiment, the tetrahydrocannabinol concentration in a 1 gram fraction of at least 10 gram sample of said homogeneous composition, is within 15% of the tetrahydrocannabinol concentration in a separate 1 gram fraction of said lo gram sample, within 13%, within 11%, within 9%, within 7%, within 5%, within 3%, within 2% or within 1% and the cannabidiol concentration in a 1 gram fraction of at least 10 gram sample of said homogeneous composition, is within 15% of the cannabidiol concentration in a separate 1 gram fraction of said 10 gram sample, within 13%, within 11%, within 9%, within 7%, within 5%, within 3%, within 2% or within 1%.

According to an embodiment, said homogeneous composition comprises at least 0.5 wt % terpene and the terpene concentration in a 1 gram fraction of at least 10 gram sample of said homogeneous composition, is within 15% of the terpene concentration in a separate 1 gram fraction of said 10 gram sample within 13%, within 11%, within 9%, within 7%, within 5%, within 3%, within 2% or within 1%.

According to an embodiment, the bulk density of said composition is greater than 0.02 gram/milliliter (gr/ml), greater than 0.04 gr/ml, greater than 0.06 gr/ml, greater than 0.08 gr/ml, greater than 0.1 gr/ml or greater than 0.12 gr/ml. According to an embodiment, the bulk density of said composition is less than 0/6 gr/ml, less than 0.5 gr/ml, less than 0.4 gr/ml, less than 0.3 gr/ml, less than 0.25 gr/ml or less than 0.2 gr/ml. According to an embodiment, the bulk density of said composition is greater than 0.02 gram/milliliter and less than 0.6 gram/milliliter.

Further provided is a product containing said homogeneous composition. Further provided is a cannabis cigarette comprising said homogeneous composition. According to an embodiment, said cannabis cigarette burns smoothly during smoking and as a result, according to an embodiment, said cannabis cigarette doesn't require re-lighting during smoking.

According to an embodiment, said cigarette contains at least 0.1 gr of said homogeneous composition, at least 0.3 gr or at least 0.5 gr. According to an embodiment, said cigarette comprises less than 2 gr of said homogeneous composition, less than 1.5 gr, less than 1.2 gr or less than 1 gr.

According to an embodiment, said cigarette contains a top fraction and a bottom fraction and the concentration of a major component in a 0.1 gram sample from the top fraction of the cigarette is within 15% of the concentration of said major component in a 0.1 gram sample from the bottom fraction of the cigarette, within 13%, within 11%, within 9%, within 7%, within 5%, within 3%, within 2% or within 1%.

According to an embodiment, said cigarette contains a top fraction and a bottom fraction and the concentration of tetrahydrocannabinol in a 0.1 gram sample from the top fraction of the cigarette is within 15% of tetrahydrocannabinol concentration in a 0.1 gram sample from the bottom fraction of the cigarette, within 13%, within 11%, within 9%, within 7%, within 5%, within 3%, within 2% or within 1%. According to an embodiment, said cigarette contains a top fraction and a bottom fraction and the concentration of cannabidiol in a 0.1 gram sample from the top fraction of the cigarette is within 15% of cannabidiol concentration in a 0.1 gram sample from the bottom fraction of the cigarette, within 13%, within 11%, within 9%, within 7%, within 5%, within 3%, within 2% or within 1%.

According to an embodiment, said cigarette contains a top fraction and a bottom fraction, the concentration of tetrahydrocannabinol in a 0.1 gram sample from the top fraction of the cigarette is within 15% of tetrahydrocannabinol concentration in a 0.1 gram sample from the bottom fraction of the cigarette, within 13%, within 11%, within 9%, within 7%, within 5%, within 3%, within 2% or within 1% and the concentration of cannabidiol in a 0.1 gram sample from the top fraction of the cigarette is within 15% of cannabidiol concentration in a 0.1 gram sample from the bottom fraction of the cigarette, within 13%, within 11%, within 9%, within 7%, within 5%, within 3%, within 2% or within 1%.

Further provided is a vaporizer cartridge containing said homogeneous composition.

According to a further aspect of some embodiments of the present invention there is provided a homogeneous tablet comprising tetrahydrocannabinol (THC) and cannabidiol (CBD), wherein
(i) tetrahydrocannabinol and cannabidiol and cannabidiol concentrations are:
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) More than 36 wt % tetrahydrocannabinol and 0.01-3 wt % cannabidiol;
(u) More than 36 wt % tetrahydrocannabinol and 3-6 wt % cannabidiol; or
(v) More than 36 wt % tetrahydrocannabinol and more than-6 wt % cannabidiol;
(i) said tablet moisture content is greater than 1 wt % and less than 20 wt %;
(ii) said tablet bulk density is at least 0.1 gram/milliliter and less than 1.2 gram/milliliter;
(iv) the tetrahydrocannabinol concentration in a fraction of said tablet, is within 15% of the tetrahydrocannabinol concentration in a separate fraction of said tablet;
(v) the cannabidiol concentration in a fraction of said tablet, is within 15% of the cannabidiol concentration in a separate fraction of said tablet; and
(vi) said tablet comprises at least 70 wt % cannabis plant material.

According to an embodiment, said homogeneous tablet comprises at least 0.01 wt % tetrahydrocannabinol, at least 2 wt % or at least 4 wt %. According to an embodiment, said homogeneous composition comprises less than 6 wt % tetrahydrocannabinol, less than 4 wt % or less than 2 wt %. According to an embodiment, said homogeneous composition comprises at least 0.1 wt % cannabidiol, at least 2 wt % or at least 4 wt %. According to an embodiment, said homogeneous composition comprises less than 6 wt % cannabidiol, less than 4 wt % or less than 2 wt %.

According to an embodiment, said homogeneous tablet comprises 0.01-6 wt % tetrahydrocannabinol and 6-12 wt % cannabidiol. According to an embodiment, said homogeneous tablet comprises at least 0.01 wt % tetrahydrocannabinol, at least 2 wt % or at least 4 wt %. According to an embodiment, said homogeneous tablet comprises less than 6 wt % tetrahydrocannabinol, less than 4 wt % or less than 2 wt %. According to an embodiment, said homogeneous tablet comprises at least 6 wt % cannabidiol, at least 8 wt % or at least 10 wt %. According to an embodiment, said homogeneous tablet comprises less than 12 wt % cannabidiol, less than 10 wt % or less than 8 wt %.

According to an embodiment, said homogeneous tablet comprises 0.01-6 wt % tetrahydrocannabinol and 12-18 wt % cannabidiol. According to an embodiment, said homogeneous tablet comprises at least 0.1 wt % tetrahydrocannabinol, at least 2 wt % or at least 4 wt %. According to an embodiment, said homogeneous tablet comprises less than 6 wt % tetrahydrocannabinol, less than 4 wt % or less than 2 wt %. According to an embodiment, said homogeneous tablet comprises at least 12 wt % cannabidiol, at least 14 wt % or at least 16 wt %. According to an embodiment, said homogeneous tablet comprises less than 18 wt % cannabidiol, less than 16 wt % or less than 14 wt %.

According to an embodiment, said homogeneous tablet comprises 0.01-6 wt % tetrahydrocannabinol and 18-24 wt % cannabidiol. According to an embodiment, said homogeneous tablet comprises at least 0.01 wt % tetrahydrocannabinol, at least 2 wt % or at least 4 wt %. According to an embodiment, said homogeneous tablet comprises less than 6 wt % tetrahydrocannabinol, less than 4 wt % or less than 2 wt %. According to an embodiment, said homogeneous tablet comprises at least 18 wt % cannabidiol, at least 20 wt % or at least 22 wt %. According to an embodiment, said homogeneous tablet comprises less than 24 wt % cannabidiol, less than 22 wt % or less than 20 wt %.

According to an embodiment, said homogeneous tablet comprises 0.01-6 wt % tetrahydrocannabinol and more than 24 wt % cannabidiol.

According to an embodiment, said homogeneous tablet comprises at least 0.01 wt % tetrahydrocannabinol, at least 2 wt % or at least 4 wt %. According to an embodiment, said homogeneous tablet comprises less than 6 wt % tetrahydrocannabinol, less than 4 wt % or less than 2 wt %. According to an embodiment, said homogeneous tablet comprises at least 24 wt % cannabidiol, at least 26 wt %, at least28 wt % or at least 30 wt %.

According to an embodiment, said homogeneous tablet comprises 6-12 wt % tetrahydrocannabinol and 0.01-6 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 0.1 wt % cannabidiol, at least 2 wt % or at least 4 wt %. According to an embodiment, said homogeneous tablet comprises less than 6 wt % cannabidiol, less than 4 wt % or less than 2 wt %.

According to an embodiment, said homogeneous tablet comprises 6-12 wt % tetrahydrocannabinol and 6-12 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 6 wt % cannabidiol, at least 8 wt % or at least 10 wt %. According to an embodiment, said homogeneous tablet comprises less than 612 wt % cannabidiol, less than 10 wt % or less than 8 wt %.

According to an embodiment, said homogeneous tablet comprises 6-12 wt % tetrahydrocannabinol and 12-18 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 12 wt % cannabidiol, at least 14 wt % or at least 16 wt %. According to an embodiment, said homogeneous tablet comprises less than 18 wt % cannabidiol, less than 16 wt % or less than 14 wt %.

According to an embodiment, said homogeneous tablet comprises 6-12 wt % tetrahydrocannabinol and 18-24 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 18 wt % cannabidiol, at least 20 wt % or at least 22 wt %. According to an embodiment, said homogeneous tablet comprises less than 24 wt % cannabidiol, less than 22 wt % or less than 20 wt %.

According to an embodiment, said homogeneous tablet comprises 6-12wt % tetrahydrocannabinol and more than 24 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 24 wt % cannabidiol, at least 26 wt %, at least28 wt % or at least 30 wt %.

According to an embodiment, said homogeneous tablet comprises 12-18 wt % tetrahydrocannabinol and 0.01-6 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 0.1 wt % cannabidiol, at least 2 wt % or at least 4 wt %. According to an embodiment, said homogeneous tablet comprises less than 6 wt % cannabidiol, less than 4 wt % or less than 2 wt %.

According to an embodiment, said homogeneous tablet comprises 12-18 wt % tetrahydrocannabinol and 6-12 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 6 wt % cannabidiol, at least 8 wt % or at least 10 wt %. According to an embodiment, said homogeneous tablet comprises less than 12 wt % cannabidiol, less than 10 wt % or less than 8 wt %.

According to an embodiment, said homogeneous tablet comprises 12-18 wt % tetrahydrocannabinol and 12-18 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 12 wt % cannabidiol, at least 14 wt % or at least 16 wt %. According to an embodiment, said homogeneous tablet comprises less than 18 wt % cannabidiol, less than 16 wt % or less than 14 wt %.

According to an embodiment, said homogeneous tablet comprises 12-18 wt % tetrahydrocannabinol and 18-24 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 18 wt % cannabidiol, at least 20 wt % or at least 22 wt %. According to an embodiment, said homogeneous tablet comprises less than 24 wt % cannabidiol, less than 22 wt % or less than 20 wt %.

According to an embodiment, said homogeneous tablet comprises 12-18wt % tetrahydrocannabinol and more than 24 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 24 wt % cannabidiol, at least 26 wt %, at least28 wt % or at least 30 wt %. According to an embodiment, said homogeneous tablet comprises 18-24 wt % tetrahydrocannabinol and 0.1-6 wt % cannabidiol. According to an embodiment, said homogeneous tablet comprises at least 18 wt % tetrahydrocannabinol, at least 20 wt % or at least 22 wt %. According to an embodiment, said homogeneous tablet comprises less than 24 wt % tetrahydrocannabinol, less than 22 wt % or less than 20 wt %. According to an embodiment, said homogeneous tablet comprises at least 0.1 wt % cannabidiol, at least 2 wt % or at least 4 wt %. According to an embodiment, said homogeneous tablet comprises less than 6 wt % cannabidiol, less than 4 wt % or less than 2 wt %.

According to an embodiment, said homogeneous tablet comprises 18-24 wt % tetrahydrocannabinol and 6-12 wt % cannabidiol. According to an embodiment, said homogeneous tablet comprises at least 18 wt % tetrahydrocannabinol, at least 20 wt % or at least 22 wt %. According to an embodiment, said homogeneous tablet comprises less than 24 wt % tetrahydrocannabinol, less than 22 wt % or less than 20 wt %. According to an embodiment, said homogeneous tablet comprises at least 6 wt % cannabidiol, at least 8 wt % or at least 10 wt %. According to an embodiment, said homogeneous tablet comprises less than 12 wt % cannabidiol, less than 10 wt % or less than 8 wt %.

According to an embodiment, said homogeneous tablet comprises 18-24 wt % tetrahydrocannabinol and 12-18 wt % cannabidiol. According to an embodiment, said homogeneous tablet comprises at least 18 wt % tetrahydrocannabinol, at least 20 wt % or at least 22 wt %. According to an embodiment, said homogeneous tablet comprises less than 24 wt % tetrahydrocannabinol, less than 22 wt % or less than 20 wt %. According to an embodiment, said homogeneous tablet comprises at least 12 wt % cannabidiol, at least 14 wt % or at least 16 wt %. According to an embodiment, said homogeneous tablet comprises less than 18 wt % cannabidiol, less than 16 wt % or less than 14 wt %.

According to an embodiment, said homogeneous tablet comprises 18-24 wt % tetrahydrocannabinol and 18-24 wt % cannabidiol. According to an embodiment, said homogeneous tablet comprises at least 18 wt % tetrahydrocannabinol, at least 20 wt % or at least 22 wt %. According to an embodiment, said homogeneous tablet comprises less than 24 wt % tetrahydrocannabinol, less than 22 wt % or less than 20 wt %. According to an embodiment, said homogeneous tablet comprises at least 18 wt % cannabidiol, at least 20 wt % or at least 22 wt %. According to an embodiment, said homogeneous tablet comprises less than 24 wt % cannabidiol, less than 22 wt % or less than 20 wt %. According to an embodiment, said homogeneous tablet comprises 18-24 wt % tetrahydrocannabinol and more than 24 wt % cannabidiol. According to an embodiment, said homogeneous tablet comprises at least 18 wt % tetrahydrocannabinol, at least 20 wt % or at least 22 wt %. According to an embodiment, said homogeneous tablet comprises less than 24 wt % tetrahydrocannabinol, less than 22 wt % or less than 20 wt %. According to an embodiment, said homogeneous tablet comprises at least 24 wt % cannabidiol, at least 26 wt %, at least28 wt % or at least 30 wt %.

According to an embodiment, said homogeneous tablet comprises more than 24 wt % tetrahydrocannabinol and 0.01-6 wt % cannabidiol. According to an embodiment, said homogeneous tablet comprises at least 24 wt % tetrahydrocannabinol, at least 26 wt %, at least 28 wt % or at least 30 wt %. According to an embodiment, said homogeneous tablet comprises at least 0.1 wt % cannabidiol, at least 2 wt % or at least 4 wt %. According to an embodiment, said homogeneous tablet comprises less than 6 wt % cannabidiol, less than 4 wt % or less than 2 wt %.

According to an embodiment, said homogeneous tablet comprises more than 24 wt % tetrahydrocannabinol and 6-12 wt % cannabidiol. According to an embodiment, said homogeneous tablet comprises at least 24 wt % tetrahydrocannabinol, at least 26 wt %, at least 28 wt % or at least 30 wt %. According to an embodiment, said homogeneous tablet comprises at least 6 wt % cannabidiol, at least 8 wt % or at least 10 wt %. According to an embodiment, said homogeneous tablet comprises less than 12 wt % cannabidiol, less than 10 wt % or less than 8 wt %.

According to an embodiment, said homogeneous tablet comprises more than 24 wt % tetrahydrocannabinol and 12-18 wt % cannabidiol. According to an embodiment, said homogeneous tablet comprises at least 24 wt % tetrahydrocannabinol, at least 26 wt %, at least28 wt % or at least 30 wt %. According to an embodiment, said homogeneous tablet comprises at least 12 wt % cannabidiol, at least 14 wt % or at least 16 wt %. According to an embodiment, said homogeneous tablet comprises less than 18 wt % cannabidiol, less than 16 wt % or less than 14 wt %.

According to an embodiment, said homogeneous tablet comprises more than 24 wt % tetrahydrocannabinol and 18-24 wt % cannabidiol. According to an embodiment, said homogeneous tablet comprises at least 24 wt % tetrahydrocannabinol, at least 26 wt %, at least28 wt % or at least 30 wt %. According to an embodiment, said homogeneous tablet comprises at least 18 wt % cannabidiol, at least 20 wt % or at least 22 wt %. According to an embodiment, said homogeneous tablet comprises less than 24 wt % cannabidiol, less than 22 wt % or less than 20 wt %.

According to an embodiment, said homogeneous tablet comprises more than 24 wt % tetrahydrocannabinol and more than 24 wt % cannabidiol. According to an embodiment, said homogeneous tablet comprises at least 24 wt % tetrahydrocannabinol, at least 26 wt %, at least28 wt % or at least 30 wt %. According to an embodiment, said homogeneous tablet comprises at least 24 wt % cannabidiol, at least 26 wt %, at least28 wt % or at least 30 wt %.

According to an embodiment, said homogeneous tablet comprises 24-30 wt % tetrahydrocannabinol and 0.01-6 wt % cannabidiol. According to an embodiment, said homogeneous tablet comprises at least 24 wt % tetrahydrocannabinol, at least 26 wt %, at least28 wt % or at least 30 wt %. According to an embodiment, said homogeneous tablet comprises at least 0.01 wt % cannabidiol, at least 2 wt %, at least4 wt % or at least 6 wt %.

According to an embodiment, said homogeneous tablet comprises 24-30 wt % tetrahydrocannabinol and 6-12 wt % cannabidiol. According to an embodiment, said homogeneous tablet comprises at least 24 wt % tetrahydrocannabinol, at least 26 wt %, at least28 wt % or at least 30 wt %. According to an embodiment, said homogeneous tablet comprises at least 6 wt % cannabidiol, at least 8 wt %, at least 10 wt % or at least 12 wt %.

According to an embodiment, said homogeneous tablet comprises 24-30 wt % tetrahydrocannabinol and 12-18 wt % cannabidiol. According to an embodiment said homogeneous tablet comprises at least 24 wt % tetrahydrocannabinol, at least 26 wt %, at least28 wt % or at least 30 wt %. According to an embodiment, said homogeneous tablet comprises at least 12 wt % cannabidiol, at least 14 wt %, at least 16 wt % or at least 18 wt %.

According to an embodiment, said homogeneous tablet comprises 30-36 wt % tetrahydrocannabinol and 0.01-6 wt % cannabidiol. According to an embodiment, said homogeneous tablet comprises at least 30 wt % tetrahydrocannabinol, at least 32 wt %, at least 34 wt % or at least 36 wt %. According to an embodiment, said homogeneous tablet comprises at least 0.01 wt % cannabidiol, at least 2 wt %, at least4 wt % or at least 6 wt %.

According to an embodiment, said homogeneous tablet comprises 30-36 wt % tetrahydrocannabinol and 6-12 wt % cannabidiol. According to an embodiment, said homogeneous tablet comprises at least 30 wt % tetrahydrocannabinol, at least 32 wt %, at least 34 wt % or at least 36 wt %. According to an embodiment, said homogeneous tablet comprises at least 6 wt % cannabidiol, at least 8 wt %, at least 10 wt % or at least 12 wt %.

According to an embodiment, said homogeneous tablet comprises 30-36 wt % tetrahydrocannabinol and 12-18 wt % cannabidiol. According to an embodiment, said homogeneous tablet comprises at least 30 wt % tetrahydrocannabinol, at least 32 wt %, at least 34 wt % or at least 36 wt %. According to an embodiment, said homogeneous tablet comprises at least 12 wt % cannabidiol, at least 14 wt %, at least 16 wt % or at least 18 wt %.

According to an embodiment, said homogeneous tablet comprises more than 36 wt % tetrahydrocannabinol and 0.01-6 wt % cannabidiol. According to an embodiment, said homogeneous tablet comprises at least 0.01 wt % cannabidiol, at least 2 wt %, at least4 wt % or at least 6 wt %.

According to an embodiment, said homogeneous tablet comprises more than 36 wt % tetrahydrocannabinol and 6-12 wt % cannabidiol. According to an embodiment, said homogeneous tablet comprises at least 6 wt % cannabidiol, at least 8 wt %, at least 10 wt % or at least 12 wt %.

According to an embodiment said homogeneous tablet comprises more than 36 wt % tetrahydrocannabinol and 12-18 wt % cannabidiol. According to an embodiment, said homogeneous tablet comprises at least 12 wt % cannabidiol, at least 14 wt %, at least 16 wt % or at least 18 wt %.

According to an embodiment, said homogeneous tablet comprises more than 36 wt % tetrahydrocannabinol and more then 6 wt % cannabidiol.

According to an embodiment, said homogeneous tablet comprises more than 36 wt % tetrahydrocannabinol and 0.01-9 wt % cannabidiol. According to an embodiment, said homogeneous tablet comprises at least 36 wt % tetrahydrocannabinol. According to an embodiment, said homogeneous tablet comprises at least 0.01 wt % cannabidiol, at least 3 wt %, at least 5% or at least 9 wt %. According to an embodiment, said homogeneous tablet comprises less than 9 wt % cannabidiol, less than 5 wt % or less than 3 wt %.

According to an embodiment, said tablet composition comprises 30-36 wt % tetrahydrocannabinol and 0.01-15 wt % cannabidiol. According to an embodiment, said homogeneous tablet comprises at least 32 wt % tetrahydrocannabinol, at least 34 wt % or at least 36 wt %. According to an embodiment, said homogeneous tablet comprises less than 36 wt % tetrahydrocannabinol, less than 34 wt % or less than 32 wt %. According to an embodiment, said homogeneous tablet comprises at least 0.01 wt % cannabidiol, at least 7 wt % or at least 15 wt %. According to an embodiment, said homogeneous tablet comprises less than 15 wt % cannabidiol, less than 10 wt % or less than 3 wt %.

According to an embodiment, said homogeneous tablet comprises 24-30 wt % tetrahydrocannabinol and 0.01-18 wt % cannabidiol. According to an embodiment, said homogeneous tablet comprises at least 26 wt % tetrahydrocannabinol, at least 28 wt % or at least 30 wt %. According to an embodiment, said homogeneous tablet comprises less than 30 wt % tetrahydrocannabinol, less than 28 wt % or less than 26 wt %. According to an embodiment, said homogeneous tablet comprises at least 0.01 wt % cannabidiol, at least 10 wt % or at least 16 wt %. According to an embodiment, said homogeneous tablet comprises less than 18 wt % cannabidiol, less than 10 wt % or less than 3 wt %.

According to an embodiment, said homogeneous tablet comprises 18-24 wt % tetrahydrocannabinol and 0.01-18 wt % cannabidiol. According to an embodiment, said homogeneous tablet comprises at least 20 wt % tetrahydrocannabinol, at least 22 wt % or at least 24 wt %. According to an embodiment, said homogeneous tablet comprises less than 26 wt % tetrahydrocannabinol, less than 24 wt % or less than 22 wt %. According to an embodiment, said tablet composition comprises at least 0.01 wt % cannabidiol, at least 10 wt % or at least 16 wt %. According to an embodiment, said homogeneous tablet comprises less than 18 wt % cannabidiol, less than 10 wt % or less than 3 wt %.

According to an embodiment, said homogeneous tablet comprises 12-18 wt % tetrahydrocannabinol and 0.01-21 wt % cannabidiol. According to an embodiment, said homogeneous tablet comprises at least 12 wt % tetrahydrocannabinol, at least 14 wt % or at least 16 wt %. According to an embodiment, said homogeneous tablet comprises less than 18 wt % tetrahydrocannabinol, less than 16 wt % or less than 14 wt %. According to an embodiment, said homogeneous tablet comprises at least 0.01 wt % cannabidiol, at least 10 wt % or at least 20 wt %. According to an embodiment, said homogeneous tablet comprises less than 21 wt % cannabidiol, less than 10 wt % or less than 3 wt %.

According to an embodiment, said homogeneous tablet comprises 6-12 wt % tetrahydrocannabinol and 0.01-24 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 12 wt % tetrahydrocannabinol, less than 10 wt % or less than 8 wt %. According to an embodiment, said homogeneous tablet comprises at least 0.01 wt % cannabidiol, at least 10 wt % or at least 22 wt %. According to an embodiment, said homogeneous tablet comprises less than 24 wt % cannabidiol, less than 10 wt % or less than 3 wt %.

According to an embodiment, said homogeneous tablet comprises 0.01-6 wt % tetrahydrocannabinol and 0.01-34 wt % cannabidiol. According to an embodiment, said homogeneous tablet comprises at least 0.01 wt % tetrahydrocannabinol, at least 2 wt % or at least 4 wt %. According to an embodiment, said homogeneous tablet comprises less than 6 wt % tetrahydrocannabinol, less than 4 wt % or less than 2 wt %. According to an embodiment, said homogeneous tablet comprises at least 0.01 wt % cannabidiol, at least 15 wt % or at least 30 wt %. According to an embodiment, said homogeneous tablet comprises less than 34 wt % cannabidiol, less than 210 wt % or less than 10 wt %.

According to an embodiment, said homogeneous tablet comprises 16-24 wt % tetrahydrocannabinol and 1-7 wt % cannabidiol. According to an embodiment, said homogeneous tablet comprises at least 16 wt % tetrahydrocannabinol, at least 18 wt % or at least 20 wt %. According to an embodiment, said homogeneous tablet comprises less than 24 wt % tetrahydrocannabinol, less than 22 wt % or less than 20 wt %. According to an embodiment, said homogeneous tablet comprises at least 1 wt % cannabidiol, at least 3 wt % or at least 5 wt %. According to an embodiment, said homogeneous tablet comprises less than 7 wt % cannabidiol, less than 5 wt % or less than 3 wt %.

According to an embodiment, said homogeneous tablet comprises 11-19 wt % tetrahydrocannabinol and 0.05-5.5 wt % cannabidiol. According to an embodiment, said homogeneous tablet comprises at least 11 wt % tetrahydrocannabinol, at least 13 wt % or at least 15 wt %. According to an embodiment, said homogeneous tablet comprises less than 19 wt % tetrahydrocannabinol, less than 17 wt % or less than 15 wt %. According to an embodiment, said homogeneous tablet comprises at least 0.5 wt % cannabidiol, at least 1.5 wt % or at least 2.5 wt %. According to an embodiment, said homogeneous tablet comprises less than 5.5 wt % cannabidiol, less than 4.5 wt % or less than 3.5 wt %.

According to an embodiment, said homogeneous tablet comprises 6-14 wt % tetrahydrocannabinol and 0.02-3.8 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 14 wt % tetrahydrocannabinol, less than 12 wt % or less than 10 wt %. According to an embodiment, said homogeneous tablet comprises at least 0.5 wt % cannabidiol, at least 1 wt % or at least 1.5 wt %. According to an embodiment, said homogeneous tablet comprises less than 3.5 wt % cannabidiol, less than 2.5 wt % or less than 1.5 wt %.

According to an embodiment, said homogeneous tablet comprises 6-14 wt % tetrahydrocannabinol and 6-14 wt % cannabidiol. According to an embodiment, said homogeneous tablet comprises at least 6 wt % tetrahydrocannabinol, at least 8 wt % or at least 10 wt %. According to an embodiment, said homogeneous tablet comprises less than 14 wt % tetrahydrocannabinol, less than 12 wt % or less than 10 wt %. According to an embodiment, said homogeneous tablet comprises at least 6 wt % cannabidiol, at least 8 wt % or at least 10 wt %. According to an embodiment, said homogeneous tablet comprises less than 14 wt % cannabidiol, less than 12 wt % or less than 10 wt %.

According to an embodiment, said homogeneous tablet comprises 2.5-7.5 wt % tetrahydrocannabinol and 6-14 wt % cannabidiol. According to an embodiment, said homogeneous tablet comprises at least 2.5 wt % tetrahydrocannabinol, at least 3.5 wt % or at least 4.5 wt %. According to an embodiment, said homogeneous tablet comprises less than 7.5 wt % tetrahydrocannabinol, less than 6.5 wt % or less than 5.5 wt %. According to an embodiment, said homogeneous tablet comprises at least 6 wt % cannabidiol, at least 8 wt % or at least 10 wt %. According to an embodiment, said homogeneous tablet comprises less than 14 wt % cannabidiol, less than 12 wt % or less than 10 wt %.

According to an embodiment, said homogeneous tablet comprises 0.05-5.5 wt % tetrahydrocannabinol and 11-19 wt % cannabidiol. According to an embodiment, said homogeneous tablet comprises at least 0.05 wt % tetrahydrocannabinol, at least 1.5 wt % or at least 2.5 wt %. According to an embodiment, said homogeneous tablet comprises less than 5.5wt % tetrahydrocannabinol, less than 4.5 wt % or less than 3.5 wt %. According to an embodiment, said homogeneous tablet comprises at least 11 wt % cannabidiol, at least 13 wt % or at least 15 wt %. According to an embodiment, said homogeneous tablet comprises less than 19 wt % cannabidiol, less than 17 wt % or less than 15 wt %.

According to an embodiment, said homogeneous tablet comprises 0.01-2.5 wt % tetrahydrocannabinol and 16-24 wt % cannabidiol. According to an embodiment, said homogeneous tablet comprises at least 0.05 wt % tetrahydrocannabinol, at least 1 wt % or at least 1.5 wt %. According to an embodiment, said homogeneous tablet comprises less than 2.5 wt % tetrahydrocannabinol, less than 2 wt % or less than 1.5 wt %. According to an embodiment, said homogeneous tablet comprises at least 16 wt % cannabidiol, at least 18 wt % or at least 20 wt %. According to an embodiment, said homogeneous tablet comprises less than 24 wt % cannabidiol, less than 22 wt % or less than 20 wt %.

According to an embodiment, said homogeneous tablet comprises 0.01-0.05 wt % tetrahydrocannabinol and 20-28 wt % cannabidiol. According to an embodiment, said homogeneous tablet comprises at least 20 wt % cannabidiol, at least 22 wt % or at least 24 wt %. According to an embodiment, said homogeneous composition comprises less than 28 wt % cannabidiol, less than 26 wt % or less than 24 wt %.

According to an embodiment, said homogeneous tablet, comprises less than 1 wt % cannabinol, less than 0.8 wt %, less than 0.7 wt %, less than 0.6 wt % or less than 0.5 wt %.

According to an embodiment, the tetrahydrocannabinol comprises tetrahydrocannabinol and tetrahydrocannabinolic acid and the weight per weight ratio between tetrahydrocannabinol and tetrahydrocannabinolic acid in said tablet is in the range between 20 to 1 and 1 to 20. According to an embodiment, the weight per weight ratio between tetrahydrocannabinol and tetrahydrocannabinolic acid in said tablet is less than 18:1, less than 16:1, less than 14:1, less than 12:1, less than 10:1, less than 8:1, less than 6:1, less than 4:1, less than 2:1 or less than 1:1. According to an embodiment, the weight per weight ratio between tetrahydrocannabinol and tetrahydrocannabinolic acid in said tablet is greater than 1:20, greater than 1:18, greater than 1:16, greater than 1:14, greater than 1:12, greater than 1:10, greater than 1:8, greater than 1:6, greater than 1:4, greater than 1:2, or greater than 1:1.

According to an embodiment, the cannabinol comprises cannabinol and cannabinolic acid and the weight per weight ratio between cannabidiol and cannabidiolic acid in said tablet is in the range between 20 to 1 and 1 to 20. According to an embodiment, the weight per weight ratio between cannabidiol and cannabidiolic acid in said tablet is less than 18:1, less than 16:1, less than 14:1, less than 12:1, less than 10:1, less than 8:1, less than 6:1, less than 4:1, less than 2:1 or less than 1:1. According to an embodiment, the weight per weight ratio between cannabidiol and cannabidiolic acid in said tablet is greater than 1:20, greater than 1:18, greater than 1:16, greater than 1:14, greater than 1:12, greater than 1:10, greater than 1:8, greater than 1:6, greater than 1:4, greater than 1:2, or greater than 1:1.

According to an embodiment at least 70 wt % of the tablet comprises cannabis plant material, at least 70 wt %, least 80 wt %, least 85 wt %, least 90 wt % or at least 95 wt %. According to an embodiment at least 70 wt % of the cannabis plant material in said tablet is derived from cannabis flower (also referred to as cannabis bud), at least 75 wt %, least 80 wt %, least 85 wt %, least 90 wt % or at least 95 wt %. As used herein, derived from cannabis flower refers to being cannabis flower or being a product or processing cannabis flower, e.g. drying and/or comminuting a cannabis flower. According to an embodiment, at least a fraction of said cannabis plant material in said composition comprises dried plant material.

According to an embodiment said tablet comprises moisture. According to an embodiment, moisture content can be determined loss on drying. According to an embodiment said composition comprises at least 1 wt % moisture; at least 3 wt %, at least 5 wt %, at least 7 wt %, at least 9 wt %, or at least 11 wt %. According to an embodiment said composition comprises less than 20 wt % moisture, less than 18 wt %, less than 16 wt %, or less than 14 wt %. According to an embodiment said composition comprises between 10 wt % and 14 wt % moisture or between 11 wt % and 13 wt % moisture.

According to an embodiment, the bulk density of said cannabis tablet is greater than 0.1 gr/ml, greater than 0.3 gr/ml, greater than 0.5 gr/ml, greater than 0.6 gr/ml, greater than 0.7 gr/ml, greater than 0.8 gr/ml or greater than 0.85 gr/ml. According to an embodiment, the bulk density of said cannabis tablet is less than 1.2 gr/ml, less than 1.1 gr/ml, less than 1.05 gr/ml, less than 1.0 gr/ml, less than 0.95 gr/ml or less than 0.90 gr/ml.

According to an embodiment, said cannabis tablet, is homogeneous. As used herein, the term homogeneous refers to having similar content in various fractions of the tablet. According to an embodiment, similar content is determined by chemical analysis of a major component. According to an embodiment, the tetrahydrocannabinol concentration in a fraction of said tablet, is within 15% of the tetrahydrocannabinol concentration in a separate fraction of said tablet within 13%, within 11%, within 9%, within 7%, within 5%, within 3%, within 2% or within 1%. According to an embodiment, the cannabidiol concentration in a fraction of said tablet, is within 15% of the cannabidiol concentration in a separate fraction of said tablet, within 13%, within 11%, within 9%, within 7%, within 5%, within 3%, within 2% or within 1%.

According to an embodiment, the weight of said tablet is between 0.1 gr and 2 gr, between 0.2 gr and 1.5 gr or between 0.25 gr and 1 gr.

According to a further aspect of some embodiments of the present invention, there is provided a method for producing a homogeneous comminuted cannabis plant material composition as disclosed herein, comprising
(i) providing a tetrahydrocannabinol-comprising cannabis plant material;
(ii) providing a cannabidiol-comprising cannabis plant material;
(iii) (a) comminuting each one of said tetrahydrocannabinol-comprising cannabis plant materials, and cannabidiol-comprising cannabis plant material whereby two separate comminuted homogeneous cannabis plant materials are formed, and/or (b) comminuting one of said tetrahydrocannabinol-comprising cannabis plant materials, and said cannabidiol-comprising cannabis plant material, whereby a comminuted homogeneous cannabis plant materials and a non-comminuted cannabis plant material are obtained and extracting the non-comminuted cannabis plant material to form a cannabinoid-comprising extract; and (iv) (a) combining said two separate comminuted homogeneous cannabis plant materials of step (iii)(a) in a proportion calculated to provide said tetrahydrocannabinol and cannabidiol concentrations of said composition, and/or (b) combining said comminuted homogeneous cannabis plant material and said cannabinoid-comprising extract of step (iii)(b) in a proportion calculated to provide said tetrahydrocannabinol and cannabidiol concentrations of said homogeneous comminuted cannabis plant material composition.

According to an embodiment said provided cannabis plant material comprises less than 30 wt % moisture, less than 28 wt %, less than 26 wt %, less than 24 wt %, less than 22 wt %, less than 20 wt %, less than 18 wt %, less than 16 wt %, or less than 14 wt %. According to an embodiment said providing cannabis plant material comprises at least 1 wt % moisture; at least 3 wt %, at least 5 wt %, at least 7 wt %, at least 9 wt %, or at least 11 wt %.

According to an embodiment, said comminuting comprises maintaining said provided cannabis plant material at a temperature of less than 0° C. for at least 1 hour to form a frozen cannabis plant material. According to an embodiment said provided cannabis plant material in kept at a temperature of less than 0° C., less than minus 5° C., less than minus 10° C., less than minus 15° C, or less than minus 20° C. According to an embodiment said provided cannabis plant material in kept at a that temperature for at least 1 hour (hr) to form frozen plant material, for at least 2 hr, at least 4 hr, at least 6 hr, at least 8 hr, at least 10 hr, at least 12 hr, at least 14 hr or at least 16 hr.

According to an embodiment, said comminuting comprises comminuting said frozen cannabis plant material by pressing against a mesh screen with a sieve size smaller than 4.2 millimeters to form expelled homogeneous composition comprising comminuted cannabis plant material. According to an embodiment, said screen is shaped as a drum or a fraction of the drum. According to an embodiment, pressing is done by an oscillating unit within said drum. According to an embodiment, oscillation is conducted at a rate between 30 and 500 rounds per minute (RPM), between 50 and 450 RPM or between 100 and 400 RPM.

According to an embodiment, screen pore size is less than 4.0 mm, less than 3.8 mm, less than 3.6 mm, less than 3.4 mm, less than 3.2 mm or less than 3.0 mm. According to an embodiment, screen pore size is at least 0.3 mm, at least 0.6 mm, at least 0.8 mm, at least 1.0 mm, at least 1.2 mm, at least 1.4 mm, at least 1.6 mm, at least 1.8 mm or at least 2.0 mm.

According to an embodiment, said comminuting comprises collecting said expelled, homogeneous composition. Any form of collecting is suitable.

According to an embodiment said method comprises analyzing a sample of said collected expelled homogeneous composition. According to an embodiment said method comprises analyzing said comminuted homogeneous cannabis plant material and said cannabinoid-comprising extract and blending them in a proportion calculated to form the comminuted cannabis plant material composition.

According to an embodiment, said extracting comprises (i) optionally comminuting the non-comminuted cannabis plant material of step (iii)(b), whereby comminuted cannabis plant material is formed; (ii) contacting said non-comminuted cannabis plant material or said comminuted cannabis plant material with a liquid extractant for at least 1 minute to form cannabinoid-depleted plant material and a cannabinoid-comprising extractant; (iii) separating said cannabinoid-depleted plant material from said cannabinoid-comprising extractant to form separated cannabinoid-comprising extractant.

According to an embodiment, contacting said provided cannabis plant material or said comminuted cannabis plant material with a liquid extractant for at least 1 minute, at least 3 minutes, at least 5 minutes to form cannabinoid-depleted plant material and a cannabinoid-comprising extractant.

According to an embodiment said method comprises analyzing a sample of said cannabinoid-comprising extractant. According to an embodiment said method comprises analyzing said two separate comminuted homogeneous cannabis plant materials and blending them in a proportion calculated to form the comminuted cannabis plant material composition.

According to an embodiment, said method comprises comminuting both said cannabis plant materials and combining said two comminuted cannabis plant materials with said cannabinoid-depleted plant material in a proportion calculated to form the said composition.

According to an embodiment, said method comprises combining said comminuted cannabis plant material and said cannabinoid-comprising extract with said cannabinoid-depleted plant material in a proportion calculated to form the said composition.

According to an embodiment at least 70 wt % of said provided a tetrahydrocannabinol-comprises cannabis plant material is derived from cannabis plant flower, at least 75 wt %, least 80 wt %, least 85 wt %, least 90 wt % or at least 95 wt %. According to an embodiment at least 70 wt % of said provided a cannabidiol-comprising cannabis plant material is derived from cannabis plant flower, at least 75 wt %, least 80 wt %, least 85 wt %, least 90 wt % or at least 95 wt %.

According to an embodiment said provided tetrahydrocannabinol-comprises cannabis plant material and/or said provided cannabidiol-comprising cannabis plant material comprise moisture at more than 1 wt %.

Any rate of production is applicable. According to an embodiment, said homogeneous composition is produced at a rate of at least 1 Kg/hr, at least 10 Kg/hr, at least 100 Kg/hr or at least 1000 Kg/hr.

According to an embodiment, said method further comprises adding said homogeneous composition to a cigarettes tube or a cigarette paper, whereby a cigarette is formed. According to an embodiment, said method comprises producing at least 10 cigarettes per hour, at least 100, at least 1000 or at least 10,000.

According to an embodiment, further provided is a cannabis cigarette produced according to the method of the present invention. According to an embodiment, said cigarette contains a top fraction and a bottom fraction and the concentration of a major component in a 0.1 gram sample from the top fraction of the cigarette is within 15% of the concentration of said major component in a 0.1 gram sample from the bottom fraction of the cigarette, within 13%, within 11%, within 9%, within 7%, within 5%, within 3%, within 2% or within 1%. According to an embodiment, said cigarette contains a top fraction and a bottom fraction and the concentration of tetrahydrocannabinol in a 0.1 gram sample from the top fraction of the cigarette is within 15% of tetrahydrocannabinol concentration in a 0.1 gram sample from the bottom fraction of the cigarette, within 13%, within 11%, within 9%, within 7%, within 5%, within 3%, within 2% or within 1%. According to an embodiment, said cigarette contains a top fraction and a bottom fraction and the concentration of cannabidiol in a 0.1 gram sample from the top fraction of the cigarette is within 15% of cannabidiol concentration in a 0.1 gram sample from the bottom fraction of the cigarette, within 13%, within 11%, within 9%, within 7%, within 5%, within 3%, within 2% or within 1%.

According to an embodiment, said cigarette contains a top fraction and a bottom fraction, the concentration of tetrahydrocannabinol in a 0.1 gram sample from the top fraction of the cigarette is within 15% of tetrahydrocannabinol concentration in a 0.1 gram sample from the bottom fraction of the cigarette, within 13%, within 11%, within 9%, within 7%, within 5%, within 3%, within 2% or within 1% and the concentration of cannabidiol in a 0.1 gram sample from the top fraction of the cigarette is within 15% of cannabidiol concentration in a 0.1 gram sample from the bottom fraction of the cigarette, within 13%, within 11%, within 9%, within 7%, within 5%, within 3%, within 2% or within 1%.

According to an embodiment, further provided is a batch of cannabis cigarettes produced according to said method, e.g. 100 cigarettes, 500 cigarettes, 1000 cigarettes, 5000 cigarettes, or mow cigarettes, and the content of tetrahydrocannabinol in one of said cigarettes is within 15% of the tetrahydrocannabinol content in a separate cigarette, within 13%, within 11%, within 9%, within 7%, within 5%, within 3%, within 2% or within 1%. According to an embodiment, the content of cannabidiol in one of said cigarettes is within 15% of the cannabinoid content in a separate cigarette, within 13%, within 11%, within 9%, within 7%, within 5%, within 3%, within 2% or within 1%.

According to an embodiment, further provided is a batch of cannabis cigarettes produced according to said method, e.g. 100 cigarettes, 500 cigarettes, 1000 cigarettes, 5000 cigarettes, or mow cigarettes, and the content of tetrahydrocannabinol in one third of the batch of the cigarettes is within 15% of the tetrahydrocannabinol content in a separate third of the batch of the cigarettes, within 13%, within 11%, within 9%, within 7%, within 5%, within 3%, within 2% or within 1%. According to an embodiment, further provided is a batch of cannabis cigarettes produced according to said method, e.g. 100 cigarettes, 500 cigarettes, 1000 cigarettes, 5000 cigarettes, or mow cigarettes, and the content of cannabidiol in one third of the batch of the cigarettes is within 15% of the cannabidiol content in a separate third of the batch of the cigarettes, within 13%, within 11%, within 9%, within 7%, within 5%, within 3%, within 2% or within 1%.

According to an embodiment, said method further comprises pressing said collected homogeneous cannabis plant material to form a homogeneous cannabis tablet. According to an embodiment, said pressing is conducted in a tablet-making machine. According to an embodiment, said method comprises producing at least 10 tablets per minute, at least 100, at least 1000 or at least 10,000.

According to an embodiment, the bulk density of said formed cannabis tablet is greater than 0.1 gr/ml, greater than 0.3 gr/ml, greater than 0.5 gr/ml, greater than 0.6 gr/ml, greater than 0.7 gr/ml, greater than 0.8 gr/ml or greater than 0.85 gr/ml. According to an embodiment, the bulk density of said formed cannabis tablet is less than 1.2 gr/ml, less than 1.1 gr/ml, less than 1.05 gr/ml, less than 1.0 gr/ml, less than 0.95 gr/ml or less than 0.90 gr/ml.

According to an embodiment, tetrahydrocannabinol concentration in a fraction of said tablet, is within 15% of the tetrahydrocannabinol concentration in a separate fraction of said tablet; within 13%, within 11%, within 9%, within 7%, within 5%, within 3%, within 2% or within 1%. According to an embodiment, the cannabidiol concentration in a fraction of said tablet, is within 15% of the cannabidiol concentration in a separate fraction of said tablet, within 13%, within 11%, within 9%, within 7%, within 5%, within 3%, within 2% or within 1%.

According to an embodiment, further provided is a batch of at least 10 homogeneous cannabis tablets produced according to said method, at least 100 or at least 1000, wherein tetrahydrocannabinol content in one tablet is within 15% of the tetrahydrocannabinol content in a separate tablet, within 13%, within 11%, within 9%, within 7%, within 5%, within 3%, within 2% or within 1%.

According to an embodiment, further provided is a batch of at least 10 homogeneous cannabis tablets produced according to said method, at least 100 or at least 1000, wherein cannabidiol content in one tablet is within 15% of the cannabidiol content in a separate tablet, within 13%, within 11%, within 9%, within 7%, within 5%, within 3%, within 2% or within 1%. According to an embodiment, further provided is a batch of homogeneous cannabis tablets produced according to said method, wherein cannabinoid content in one third of the tablets is within 15% of the THC content in a separate third of the tablets, within 13%, within 11%, within 9%, within 7%, within 5%, within 3%, within 2% or within 1%

According to an embodiment, the weight of said tablet is at least 0.05 gr, at least 0.1 gr or at least 0.15 gr. According to an embodiment, the weight of said tablet is less than 1 gr, less than 0.8 gr, less than 0.6 gr or less than 0.4 gr. According to an embodiment, the cannabinoid content in said tablet is at least 2 milligrams (mg), at least 5 mg, at least 10 mg or at least 15 mg. According to an embodiment, the cannabinoid content in said tablet is less than 50 mg, less than 40 mg, less than 30 mg, or less than 20 mg.

According to an embodiment, the method further comprises heating said cannabis tablet to a temperature greater than 100° C., whereby said cannabinoid decarboxlates and/or evaporate. According to an embodiment, said heating decarboxylation and evaporation are conducted in a cannabis vaporizer.

### EXAMPLES

Examples 1: Production of homogeneous comminuted cannabis composition via blending two separate homogeneous comminuted cannabis plant materials.

Tetrahydrocannabinol-comprising cannabis flowers were dried to moisture content of 14 wt %. Separately. cannabidiol-comprising cannabis flowers were dried to moisture content of 14 wt %. The dried flowers from both plants were kept in a freezer at a temperature of minus 10° C. for 24 hours. The frozen flowers of each plant were comminuted, as such, without warming, by pressing against a screen with a pore size of 2 mm. The expelled comminuted material was collected and allowed to warm to (plus) 200° C. Then a sample of the expelled material was taken and its bulk density was determined. Bulk densities of 0.138 and 0.143 (gr/ml) were measured for the tetrahydrocannabinol-comprising cannabis and cannabidiol-comprising cannabis comminuted plant materials respectively.

The two comminuted homogeneous cannabis plant materials were blended at a proportion calculated to form 18% THC 6% CBD homogeneous comminuted cannabis plant material composition

Example 2: Flowers of tetrahydrocannabinol-comprising cannabis plant A were dried to a moisture content of 12 wt %. Separately Flowers of cannabidiol-comprising cannabis plant B were dried to a moisture content of 12 wt %. The dried flowers from each plant were kept in a freezer at a temperature of minus 10° C. for 24 hours. The frozen flowers of plant A were comminuted, as such, without warming, by pressing against a screen with a pore size of 2 mm. The expelled comminuted material was collected, allowed to warm up to (plus) 200° C. and homogenized to form comminuted homogeneous flower material A. The frozen flowers of plant B were treated similarly to form comminuted homogeneous flower material B. Each flower material was analyzed for their cannabinoids and terpenes content.

Examples 3-10: Flowers of tetrahydrocannabinol-comprising cannabis plant A were dried to a moisture content of 12 wt %. Separately, flowers of cannabidiol-comprising cannabis plant B were dried to a moisture content of 12 wt %. The dried flowers from each plant were kept in a freezer at a temperature of minus 10° C. for 24 hours. The frozen flowers of plant A were comminuted, as such, without warming, by pressing against a screen with a pore size of 2 mm. The expelled comminuted material was collected, allowed to warm up to (plus) 20° C. and homogenized to form comminuted homogeneous flower material A. The frozen flowers of plant B were treated similarly to form comminuted homogeneous flower material B. Each flower material was analyzed for its cannabinoids and terpenes content. Selected amounts of flower material A were blended with selected amounts of flower material B to form homogeneous blends. The blend was analyzed for its cannabinoids and terpenes content. Results are summarized in the following Table 1. A large variety of compositions was formed through that blending.

TABLE 1

| | Flower material A | | Flower material B | | Selected amounts | | Blend | |
|---|---|---|---|---|---|---|---|---|
| | cannabinoids content (%) | | cannabinoids content (%) | | (Kg) | | cannabinoids content (%) | |
| Example | THCa | CBDa | THCa | CBDa | A | B | THCa | CBDa |
| 3 | 24.2 | 0.1 | 11.8 | 9.6 | 0.5 | 1.5 | 15 | 7.1 |
| 4 | 24.2 | 0.1 | 11.8 | 9.6 | 1.5 | 0.5 | 21 | 4.9 |
| 5 | 15.3 | 0.1 | 7.8 | 13.3 | 0.5 | 1.5 | 9.2 | 10 |
| 6 | 15.3 | 0.1 | 7.8 | 13.3 | 1.5 | 0.5 | 13.3 | 3.3 |
| 7 | 18.4 | 0.1 | 3.2 | 15.5 | 0.5 | 1.5 | 7 | 11.6 |
| 8 | 18.4 | 0.1 | 3.2 | 15.5 | 1.5 | 0.5 | 14.4 | 3.9 |
| 9 | 8.8 | 12.2 | 14.8 | 10.2 | 0.5 | 1.5 | 13.3 | 10.6 |
| 10 | 8.8 | 12.2 | 14.8 | 10.2 | 1.5 | 0.5 | 10.2 | 12 |

Examples 11-17: Flowers of cannabis plant A were dried to a moisture content of 12 wt %. Separately, flowers of cannabis plant B were dried to a moisture content of 12 wt %. The dried flowers from each plant were kept in a freezer at a temperature of minus 10° C. for 24 hours. The frozen flowers of plant A were comminuted, as such, without warming, by pressing against a screen with a pore size of 2 mm. The expelled comminuted material was collected, allowed to warm up to (plus) 200° C. and homogenized to form comminuted homogeneous flower material A. This flower material A was analyzed for its cannabinoids and terpenes content. The frozen flowers of plant B were treated similarly to form comminuted homogeneous flower material B. A selected weight of flower material B was mixed at ambient temperature in an extraction vessel with 10 weights of 99% ethanol for 30 minutes. Then the residual flower material was filtered out. The remaining, B extract-containing ethanol was evaporated to remove 80% of its volume, forming concentrated B extract. A sample of that concentrated B extract was subjected to further evaporation to form desolventized B extract. That desolventized B extract was analyzed for its cannabinoids content. A selected amount of flower material A was thoroughly mixed with a selected amount of concentrated B extract to form a wet A+B blend. That wet blend was dried in an oven at 40° C. to form a dry A+B blend. That dry blend was analyzed for its cannabinoids content. Results are summarized in the following Table 2. Large variety of dry blends was formed, including dry blends with cannabinoids concentrations far beyond those found in cannabis flowers.

TABLE 2

| | Flower material A | | Desolventized B extract | | Selected amounts | | Dry A + B blend | |
|---|---|---|---|---|---|---|---|---|
| | cannabinoids content (%) | | cannabinoids content (%) | | (Kg) | | cannabinoids content (%) | |
| Example | THCa | CBDa | THCa | CBDa | A | B | THCa | CBDa |
| 11 | 24.2 | 0.1 | 67 | 1 | 1 | 0.35 | 35 | 0.4 |
| 12 | 1 | 16 | 4 | 64 | 1 | 0.35 | 1.5 | 28 |
| 13 | 24.2 | 0.1 | 30 | 34 | 1 | 0.25 | 25 | 7 |
| 14 | 7.8 | 13.3 | 4 | 64 | 1 | 0.3 | 7 | 25 |
| 15 | 7.8 | 13.3 | 30 | 34 | 1 | 0.3 | 13 | 18 |
| 16 | 7.8 | 13.3 | 67 | 1 | 1 | 0.3 | 21 | 10.5 |
| 17 | 14.8 | 10.2 | 30 | 34 | 1 | 0.3 | 18 | 16 |

Examples 18-24: Flowers of cannabis plant A were dried to a moisture content of 12 wt %. Separately, flowers of cannabis plant B were dried to a moisture content of 12 wt %. The dried flowers from each plant were kept in a freezer at a temperature of minus 100° C. for 24 hours. The frozen flowers of plant A were comminuted, as such, without warming, by pressing against a screen with a pore size of 2 mm. The expelled comminuted material was collected, allowed to warm up to (plus) 200° C. and homogenized to form comminuted homogeneous flower material A. This flower material A was analyzed for its cannabinoids and terpenes content. The frozen flowers of plant B were treated similarly to form comminuted homogeneous flower material B. A selected weight of flower material B was mixed at ambient temperature in an extraction vessel with 10 weights of 99% ethanol for 30 minutes. Then, the residual flower material was filtered out. The remaining, B extract-containing ethanol was evaporated to remove all ethanol content and then kept at 120° C. to complete decarboxylation, forming decarboxylated B extract. A sample of that decarboxylated B extract was analyzed for its cannabinoids content. The decarboxylated B extract was diluted by mixing each weight of it with two weights of ethanol 99% to form diluted decarboxylated B extract. A selected amount of flower material A was thoroughly mixed with a selected amount of diluted decarboxylated B extract to form a wet A+B(d) blend. That wet blend was dried in an oven at 40° C. to form a dry A+B(d) blend. That dry blend was analyzed for its cannabinoids content. Results are summarized in the following Table 3. Large variety of dry blends was formed, including dry blends with cannabinoids concentrations far beyond those found in cannabis flowers and various ratios between decarboxylated and non-decarboxylated cannabinoids.

TABLE 3

| | Flower material A | | Decarboxylated B extract | | Selected amounts | | Dry A + B (d) blend | | | |
|---|---|---|---|---|---|---|---|---|---|---|
| | cannabinoids content (%) | | cannabinoids content (%) | | (Kg) | | cannabinoids content (%) | | | |
| Ex. | THCa | CBDa | THC | CBD | A | B | THCa | THC | CBDa | CBD |
| 18 | 24.2 | 0.1 | 67 | 1 | 1 | 0.35 | 18 | 17 | 0.1 | 0.3 |
| 19 | 1 | 16 | 4 | 64 | 1 | 0.35 | 1 | 1 | 12 | 17 |
| 20 | 24.2 | 0.1 | 30 | 34 | 1 | 0.25 | 19 | 6 | 0.1 | 7 |
| 21 | 7.8 | 13.3 | 4 | 64 | 1 | 0.3 | 6 | 1 | 10 | 15 |
| 22 | 7.8 | 13.3 | 30 | 34 | 1 | 0.3 | 6 | 7 | 10 | 8 |
| 23 | 7.8 | 13.3 | 67 | 1 | 1 | 0.3 | 6 | 15 | 10 | 0.2 |
| 24 | 14.8 | 10.2 | 30 | 34 | 1 | 0.3 | 11 | 7 | 8 | 8 |

Example 25: Production of a cannabis tablet.

10 gr of blends prepared according to Examples 2-23 were introduced into a tablet-making machine. 50 tablets were formed. The tablets sink when put in 95 wt % ethanol solution and float when put in water.

Examples 26-37

The methods of examples 2-23 were used to produce blends of compositions summarized in the following Table 4:

TABLE 4

Cannabinoids concentration (%)

| Example # | THCa | THC | CBDa | CBD |
|---|---|---|---|---|
| 26 | 3 | 1 | 1 | 5 |
| 27 | 1 | 2 | 8 | 3 |
| 28 | 5 | 0.5 | 6 | 10 |
| 29 | 3 | 3 | 12 | 7 |
| 30 | 0.5 | 5 | 11 | 9 |
| 31 | 5 | 0.5 | 16 | 8 |
| 32 | 8 | 3 | 9 | 2 |
| 33 | 7 | 4 | 15 | 7 |
| 34 | 16 | 4 | 12 | 3 |
| 38 | 22 | 6 | 5 | 2 |
| 36 | 24 | 8 | 4 | 1 |
| 37 | 25 | 9 | 5 | 2 |

Thus, the scope of the invention shall include all modifications and variations that may fall within the scope of the attached claims. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A homogeneous cannabis plant material composition comprising tetrahydrocannabinol (THC) and cannabidiol (CBD), wherein the plant material is in the form of comminuted particles and wherein
(i) tetrahydrocannabinol and cannabidiol concentrations are selected from the group consisting of
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) more than 36 wt % tetrahydrocannabinol and 0.01-3 wt % cannabidiol;
(u) more than 36 wt % tetrahydrocannabinol and 3-6 wt % cannabidiol; and
(v) more than 36 wt % tetrahydrocannabinol and more than6 wt % cannabidiol; and wherein
(ii) at least 70 wt % of the comminuted cannabis plant material is of a size greater than 0.3 millimeter and less than 4.2 millimeters;
(iii) the composition comprises at least 1 wt % moisture and less than 20 wt % moisture;
(iv) the homogeneous composition, when separated into at least a 10 gram sample, contains a tetrahydrocannabinol concentration in a 1 gram fraction of the at least 10 gram sample within 15% of the tetrahydrocannabinol concentration in a separate 1 gram fraction of said at least 10 gram sample; and
(v) and the homogeneous composition, when separated into at least a 10 gram sample, contains a—cannabidiol concentration in a 1 gram fraction of the at least 10 gram sample within 15% of the cannabidiol concentration in a separate 1 gram fraction of said at least 10 gram sample.

2. The homogeneous composition of claim 1, wherein the tetrahydrocannabinol comprises tetrahydrocannabinol and tetrahydrocannabinolic acid and wherein the weight per weight ratio between tetrahydrocannabinol and tetrahydrocannabinolic acid is in the range between 20 to 1 and 1 to 20 and wherein the cannabidiol comprises cannabidiol and cannabidiolic acid and wherein the weight per weight ratio between cannabidiol and cannabidiolic acid is in the range between 20 to 1 and 1 to 20.

3. The homogeneous composition of claim 1, having a bulk density greater than 0.02 gram/milliliter and less than 0.6 gram/milliliter.

4. A product comprising the homogeneous composition of claim 1, wherein the product is selected from the group consisting of a cigarette and a vaporizer cartridge.

5. A homogeneous tablet, comprising tetrahydrocannabinol (THC) and cannabidiol (CBD), wherein
(i) tetrahydrocannabinol and cannabidiol and cannabidiol concentrations are:
(a) 0.01-6 wt % tetrahydrocannabinol and 18-24 wt % cannabidiol;
(b) 0.01-6 wt % tetrahydrocannabinol and 24-28 wt % cannabidiol;
(c) 0.01-6 wt % tetrahydrocannabinol and more than 28 wt % cannabidiol;
(d) 6-12 wt % tetrahydrocannabinol and 12-18 wt % cannabidiol;
(e) 6-12 wt % tetrahydrocannabinol and 18-24 wt % cannabidiol;
(f) 6-12 wt % tetrahydrocannabinol and more than 24 wt % cannabidiol;
(g) 12-18 wt % tetrahydrocannabinol and 6-12 wt % cannabidiol;
(h) 12-18 wt % tetrahydrocannabinol and 12-18 wt % cannabidiol;
(i) 12-18 wt % tetrahydrocannabinol and 18-24 wt % cannabidiol;
(j) 12-18 wt % tetrahydrocannabinol and more than 24 wt % cannabidiol;
(k) 18-24 wt % tetrahydrocannabinol and 6-12 wt % cannabidiol;
(l) 18-24 wt % tetrahydrocannabinol and 12-18 wt % cannabidiol;
(m) 18-24 wt % tetrahydrocannabinol and 18-24 wt % cannabidiol;
(n) 18-24 wt % tetrahydrocannabinol and more than 24 wt % cannabidiol;
(o) 24-30 wt % tetrahydrocannabinol and 6-12 wt % cannabidiol;
(p) 24-30 wt % tetrahydrocannabinol and 12-18 wt % cannabidiol;
(q) 30-36 wt % tetrahydrocannabinol and 0.01-6 wt % cannabidiol;
(r) 30-36 wt % tetrahydrocannabinol and 6-12 wt % cannabidiol;
(s) 30-36 wt % tetrahydrocannabinol and 12-18 wt % cannabidiol;
(t) More than 36 wt % tetrahydrocannabinol and 0.01-3 wt % cannabidiol;
(u) More than 36 wt % tetrahydrocannabinol and 3-6 wt % cannabidiol; or
(v) More than 36 wt % tetrahydrocannabinol and more than-6 wt % cannabidiol;
(ii) said tablet moisture content is greater than 1 wt % and less than 20 wt %;
(iii) said tablet bulk density is at least 0.1 gram/milliliter and less than 1.2 gram/milliliter;
(iv) the tetrahydrocannabinol concentration in a fraction of said tablet, is within 15% of the tetrahydrocannabinol concentration in a separate fraction of said tablet;
(v) the cannabidiol concentration in a fraction of said tablet, is within 15% of the cannabidiol concentration in a separate fraction of said tablet; and
(vi) said tablet comprises at least 70 wt % cannabis plant material.

6. The homogeneous tablet of claim 5, wherein the cannabidiol comprises cannabidiol and cannabidiolic acid and wherein the weight per weight ratio between cannabidiol and cannabidiolic acid is in the range between 20 to 1 and 1 to 20 and wherein the tetrahydrocannabinol comprises tetrahydrocannabinol and tetrahydrocannabinolic acid and wherein a weight per weight ratio between tetrahydrocannabinol and tetrahydrocannabinolic acid is in the range between 20 to 1 and 1 to 20.

7. A method for producing the homogeneous cannabis plant material composition, of claim 1 comprising
(i) providing a tetrahydrocannabinol-comprising cannabis plant material;
(ii) providing a cannabidiol-comprising cannabis plant material;
(iii) (a) comminuting said tetrahydrocannabinol-comprising cannabis plant material, and said cannabidiol-comprising cannabis plant material whereby two separate comminuted homogeneous cannabis plant materials are formed, and/or (b) comminuting one of said tetrahydrocannabinol-comprising cannabis plant materials, and said cannabidiol-comprising cannabis plant material, whereby a comminuted homogeneous cannabis plant material and a non-comminuted cannabis plant material are obtained and extracting the non-comminuted cannabis plant material to form a cannabinoid-comprising extract; and
(iv) (a) combining said two separate comminuted homogeneous cannabis plant materials of step (iii)(a) in a proportion calculated to provide said tetrahydrocannabinol and cannabidiol concentrations of said composition, and/or (b) combining said comminuted homogeneous cannabis plant material and said cannabinoid-comprising extract of step (iii)(b) in a proportion calculated to provide said tetrahydrocannabinol and cannabidiol concentrations of said homogeneous comminuted cannabis plant material composition.

8. The method of claim 7, wherein said extracting comprises
(i) optionally comminuting the non-comminuted cannabis plant material of step (iii)(b), whereby comminuted cannabis plant material is formed;
(ii) contacting said non-comminuted cannabis plant material or said comminuted cannabis plant material with a liquid extractant for at least 1 minute to form cannabinoid-depleted plant material and a cannabinoid-comprising extractant, and
(iii) separating said cannabinoid-depleted plant material from said cannabinoid-comprising extractant to form separated cannabinoid-comprising extractant.

9. The method of claim 7, further comprising combining said two comminuted cannabis plant materials or said comminuted cannabis plant material and said cannabinoid-comprising extract with said cannabinoid-depleted plant material in a proportion calculated to form the said composition.

10. The method of claim 7, comprising blending said comminuted cannabis plant material and said cannabinoid-comprising extract with said cannabinoid-depleted plant material in a proportion calculated to form the said composition.

7. method of claim 7, further comprising adding said homogeneous composition to a cigarettes tube or a cigarette paper, whereby a cigarette is formed.

12. A cannabis cigarette produced according to the method of claim 11, wherein said cigarette contains a top fraction and a bottom fraction and the concentration of tetrahydrocannabinol in a 0.1 gram sample from the top fraction of the cigarette is within 15% of tetrahydrocannabinol concentration in a 0.1 gram sample from the bottom fraction of the cigarette and wherein cannabidiol in a 0.1 gram sample from the top fraction of the cigarette is within 15% of cannabidiol concentration in a 0.1 gram sample from the bottom fraction of the cigarette.

13. A batch of cannabis cigarettes produced according to the method of claim 11, wherein the content of tetrahydrocannabinol in one third of the batch of the cigarettes is within 15% of the tetrahydrocannabinol content in a separate third of the batch of the cigarettes and wherein the content of cannabidiol in one third of the batch of the cigarettes is within 15% of the cannabidiol content in a separate third of the batch of the cigarettes.

14. The method of claim 7, further comprising pressing said homogeneous comminuted composition to form a homogeneous cannabis tablet.

15. A homogeneous cannabis tablet produced according to the method of claim 7, wherein said tablet has a bulk density of at least 0.1 gram/milliliter and less than 1.2 gram/milliliter.
